(19) United States
(12) Patent Application Publication
(10) Pub. No.: US 2022/0146526 A1
(43) Pub. Date: May 12, 2022

(54) METHOD FOR EVALUATING SAMPLE, ANALYSIS METHOD, METHOD FOR DETECTING DEGRADED SAMPLE, MARKER FOR DETECTING DEGRADED BLOOD PLASMA SAMPLE, AND MARKER FOR DETECTING DEGRADED SERUM SAMPLE

(71) Applicant: SHIMADZU CORPORATION, Kyoto (JP)

(72) Inventors: Yumi UNNO, Kyoto (JP); Shuichi KAWANA, Kyoto (JP); Yutaka AOKI, Kyoto (JP); Junichi MASUDA, Kyoto (JP); Hirotaka FUJIMOTO, Kyoto (JP); Tsuyoshi NAKANISHI, Kyoto (JP)

(73) Assignee: SHIMADZU CORPORATION, Kyoto (JP)

(21) Appl. No.: 17/437,720

(22) PCT Filed: Jan. 27, 2020
(86) PCT No.: PCT/JP2020/002842

§ 371 Date: Sep. 9, 2021

(30) Foreign Application Priority Data
May 9, 2019 (JP) 2019-089363

(51) Int. Cl. G01N 33/68; G01N 33/70; G01N 33/92; G01N 30/72

(52) U.S. Cl. CPC G01N 33/6815; G01N 33/70; G01N 30/7206; G01N 30/7233; G01N 33/92

(57) ABSTRACT

A method for evaluating a sample includes obtaining a blood plasma sample prepared from human blood, conducting detection of a predetermined molecule in the blood plasma sample, and evaluating the quality of the blood plasma sample based on the intensity of the molecule acquired by the detection.

### TECHNICAL FIELD

The present invention relates to a method for evaluating a sample, an analysis method, a method for detecting a degraded sample, a marker for detecting a degraded blood plasma sample, and a marker for detecting a degraded serum sample.

### BACKGROUND ART

Methods for acquiring information on the assessment of the risk of suffering from disease, the diagnosis, or the prediction of prognosis by detecting molecules contained in blood plasma or serum by analysis such as mass spectrometry have been studied. In this analysis, the results acquired may vary depending on the conditions under which the blood plasma sample or serum sample is prepared or stored.

In Non-Patent Document 1, fluctuations in the amount of metabolites detected depending on the conditions for preparation or storage of blood plasma samples or serum samples are observed by capillary electrophoresis-mass spectrometry. In Non-Patent Document 2, similar observation is conducted by gas chromatography/mass spectrometry and liquid chromatography/mass spectrometry. Non-Patent Document 3 and Non-Patent Document 4 propose to search for molecules of which the detected amount fluctuates depending on the conditions for preparation or storage as markers for quality evaluation.

### PRIOR ART DOCUMENTS

### Non-Patent Documents

Non-Patent Document 1: Hirayama A, Sugimoto M, Suzuki A, Hatakeyama Y, Enomoto A, Harada S, Soga T, Tomita M, Takebayashi T. “Effects of processing and storage conditions on charged metabolomic profiles in blood.” Electrophoresis, (Germany), Wiley-VCH, September 2015, Volume 36, Issue 18, p.2148-2155
Non-Patent Document 2: Nishiumi S, Suzuki M, Kobayashi T, Yoshida M. “Differences in metabolite profiles caused by pre-analytical blood processing procedures.” Journal of bioscience and bioengineering, (Japan), Society for Bioscience and Bioengineering, Japan, May 2018, Volume 125, Issue 5, p.613-618
Non-Patent Document 3: Kamlage B, Maldonado SG, Bethan B, Peter E, Schmitz O, Liebenberg V, Schatz P. “Serum metabolomics reveals γ-glutamyl dipeptides as biomarkers for discrimination among different forms of liver disease.” Clinical Chemistry (USA), American Association For Clinical Chemistry, February 2014, Volume 60, Issue 2, p.399-412
Non-Patent Document 4: Supervised by Kasuga and 3 others, written by Minegishi and 18 others, “Report on handling of biological samples for omics research”, [online], Aug. 1, 2017, Japan Agency for Medical Research and Development, [Searched on Mar. 22, 2019], Internet (https://www.biobank.amed.go.jp/2017/08/08/content/pdf/medical/omicsreport0810.pdf)

### SUMMARY OF THE INVENTION

### Problems to be Solved by the Invention

It is desirable to accurately evaluate the quality of a blood plasma sample or a serum sample using a proper marker.

### Means for Solving the Problems

A first aspect of the present invention relates to a method for evaluating a sample, which includes: obtaining a blood plasma sample prepared from human blood; conducting detection of at least one molecule selected from the group consisting of 1,6-anhydroglucose, 1-hexadecanol, 2-aminobutyric acid, 2-ketobutyric acid, 2′-deoxyuridine, 2-hydroxyisocaproic acid, 2-hydroxypyridine, 3-aminoisobutyric acid, 3-sulfinoalanine, 3-phenyllactic acid, 4-aminobutyric acid, 4-hydroxyphenyllactic acid, 4-hydroxyproline, 5-glutamylcysteine, N6-acetyllysine, N-acetylserine, S-adenosylhomocysteine, S-adenosylmethionine, aconitic acid, ascorbic acid, asparagine, aspartic acid, acetylcarnitine, azelaic acid, adenine, adenosine, adenosine monophosphate, adenosine 3′,5′-cyclic monophosphate, arachidonic acid, alanine, allantoin, argininosuccinic acid, arginine, isoleucine, inosine, indoxyl sulfate, uridine, octadecanol, ornithine, oleic acid, cabroic acid, galacturonic acid, carnitine, xanthine, xylose, kynurenine, quinolinic acid, guanosine, guanosine monophosphate, glyoxylic acid, glycolic acid, glycine, glycerol-3-phosphate, glutamine, glutamic acid, creatinine, creatine, cholic acid, succinic acid, choline, cholesterol, cystathionine, cystine, cysteine, citicoline, cytidine, cytidine monophosphate, cytosine, citrulline, dihydrouracil, dihydroxyacetone phosphate, dimethylglycine, oxalic acid, scyllo-inositol, sucrose, stearic acid, serotonin, sorbose, symmetric dimethylarginine, dopa, dopamine, docosahexaenoic acid, tryptamine, tryptophan, trehalose, nicotinamide, uric acid, paraxanthine, palmitic acid, pantothenic acid, histamine, histidine, asymmetric dimethylarginine, hydroquinone, hypoxanthine, hypoxanthine, hypotaurine, psicose, proline, boric acid, homocysteine, maleic acid, mannose, myristic acid, methionine sulfoxide, methionine sulfone, monostearin, lactitol, lactose, linoleic acid, ribulose, ribose, ribonic acid, malic acid, leucine, and uric acid in the blood plasma sample; and evaluating quality of the blood plasma sample based on intensity of the molecule acquired by the detection.

A second aspect of the present invention relates to an analysis method, which includes conducting evaluation of a blood plasma sample by the method for evaluating a sample according to the first aspect and conducting analysis of a blood plasma sample based on the evaluation.

A third aspect of the present invention relates to a method for detecting a degraded sample, which includes: obtaining a blood plasma sample prepared from human blood; and conducting detection of at least one molecule selected from the group consisting of 1,6-anhydroglucose, 1-hexadecanol, 2-aminobutyric acid, 2-ketobutyric acid, 2′-deoxyuridine, 2-hydroxyisocaproic acid, 2-hydroxypyridine, 3-aminoisobutyric acid, 3-sulfinoalanine, 3-phenyllactic acid, 4-aminobutyric acid, 4-hydroxyphenyllactic acid, 4-hydroxyproline, 5-glutamylcysteine, N6-acetyllysine, N-acetylserine, S-adenosylhomocysteine, S-adenosylmethionine, aconitic acid, ascorbic acid, asparagine, aspartic acid, acetylcarnitine, azelaic acid, adenine, adenosine, adenosine monophosphate, adenosine 3′,5′-cyclic monophosphate, arachidonic acid, alanine, allantoin, argininosuccinic acid, arginine, isoleucine, inosine, indoxyl sulfate, uridine, octadecanol, ornithine, oleic acid, cabroic acid, galacturonic acid, carnitine, xanthine, xylose, kynurenine, quinolinic acid, guanosine, guanosine monophosphate, glyoxylic acid, glycolic acid, glycine, glycerol-3-phosphate, glutamine, glutamic acid, creatinine, creatine, cholic acid, succinic acid, choline, cholesterol, cystathionine, cystine, cysteine, citicoline, cytidine, cytidine monophosphate, cytosine, citrulline, dihydrouracil, dihydroxyacetone phosphate, dimethylglycine, oxalic acid, scyllo-inositol, sucrose, stearic acid, serotonin, sorbose, symmetric dimethylarginine, dopa, dopamine, docosahexaenoic acid, tryptamine, tryptophan, trehalose, nicotinamide, uric acid, paraxanthine, palmitic acid, pantothenic acid, histamine, histidine, asymmetric dimethylarginine, hydroquinone, hypoxanthine, hypoxanthine, hypotaurine, psicose, proline, boric acid, homocysteine, maleic acid, mannose, myristic acid, methionine sulfoxide, methionine sulfone, monostearin, lactitol, lactose, linoleic acid, ribulose, ribose, ribonic acid, malic acid, leucine, and uric acid in the blood plasma sample.

A fourth aspect of the present invention relates to a marker for detecting a degraded blood plasma sample, which contains at least one molecule selected from the group consisting of 1,6-anhydroglucose, 1-hexadecanol, 2-aminobutyric acid, 2-ketobutyric acid, 2′-deoxyuridine, 2-hydroxyisocaproic acid, 2-hydroxypyridine, 3-aminoisobutyric acid, 3-sulfinoalanine, 3-phenyllactic acid, 4-aminobutyric acid, 4-hydroxyphenyllactic acid, 4-hydroxyproline, 5-glutamylcysteine, N6-acetyllysine, N-acetylserine, S-adenosylhomocysteine, S-adenosylmethionine, aconitic acid, ascorbic acid, asparagine, aspartic acid, acetylcarnitine, azelaic acid, adenine, adenosine, adenosine monophosphate, adenosine 3′,5′-cyclic monophosphate, arachidonic acid, alanine, allantoin, argininosuccinic acid, arginine, isoleucine, inosine, indoxyl sulfate, uridine, octadecanol, ornithine, oleic acid, cabroic acid, galacturonic acid, carnitine, xanthine, xylose, kynurenine, quinolinic acid, guanosine, guanosine monophosphate, glyoxylic acid, glycolic acid, glycine, glycerol-3-phosphate, glutamine, glutamic acid, creatinine, creatine, cholic acid, succinic acid, choline, cholesterol, cystathionine, cystine, cysteine, citicoline, cytidine, cytidine monophosphate, cytosine, citrulline, dihydrouracil, dihydroxyacetone phosphate, dimethylglycine, oxalic acid, scyllo-inositol, sucrose, stearic acid, serotonin, sorbose, symmetric dimethylarginine, dopa, dopamine, docosahexaenoic acid, tryptamine, tryptophan, trehalose, nicotinamide, uric acid, paraxanthine, palmitic acid, pantothenic acid, histamine, histidine, asymmetric dimethylarginine, hydroquinone, hypoxanthine, hypoxanthine, hypotaurine, psicose, proline, boric acid, homocysteine, maleic acid, mannose, myristic acid, methionine sulfoxide, methionine sulfone, monostearin, lactitol, lactose, linoleic acid, ribulose, ribose, ribonic acid, malic acid, leucine, and uric acid.

A fifth aspect of the present invention relates to a method for evaluating a sample, which includes: obtaining a serum sample prepared from human blood; conducting detection of at least one molecule selected from the group consisting of 1,6-anhydroglucose, 1-hexadecanol, 2-aminooctanoic acid, 2-aminobutyric acid, 2-ketoisovaleric acid, 2-hydroxyglutaric acid, 2-hydroxypyridine, 3-aminoisobutyric acid, 3-aminopropionic acid, β-alanine, 3-indolepropionic acid, 3-sulfinoalanine, 3-hydroxyanthranyl acid, 3-hydroxyisovaleric acid, 3-hydroxypyruvic acid, 3-hydroxypropionic acid, 3-phenyllactic acid, 4-hydroxyphenyllactic acid, 4-hydroxyproline, 5-hydroxymethyl-2-furancarboxylic acid, N6-acetyllysine, N-acetylglutamine, N-acetylserine, S-adenosylhomocysteine, aconitic acid, adipic acid, ascorbic acid, asparagine, aspartic acid, acetylcarnitine, acetylglycine, acetoacetic acid, azelaic acid, adenine, adenosine, adenosine monophosphate, adenosine 3′,5′-cyclic monophosphate, arachidonic acid, alanine, allantoin, argininosuccinic acid, arginine, allose, benzoic acid, isoleucine, inositol, inosine, uracil, uridine, eicosapentaenoic acid, erythrulose, octadecanol, ornithine, oleamide, cadaverine, cabroic acid, galacturonic acid, carnitine, carnosine, xanthine, xylitol, xylulose, xylose, kynurenine, guanosine, guanosine 3′,5′-cyclic monophosphate, glyoxylic acid, glycolic acid, glycine, glycerol-3-phosphate, glucosamine, gluconic acid, glutamic acid, glutaric acid, creatinine, creatine, cholic acid, succinic acid, choline, sarcosine, cystine, cysteine, cytidine, citramalic acid, citrulline, dihydrouracil, dihydroxyacetone phosphate, dimethylglycine, oxalic acid, scyllo-inositol, sucrose, stearic acid, serine, serotonin, sorbitol, sorbose, tyramine, tyrosine, decanoic acid, dopa, dopamine, docosahexaenoic acid, tryptophan, threonine, threonic acid, trehalose, nicotinamide, paraxanthine, valine, pantothenic acid, histidine, asymmetric dimethylarginine, hydroxylamine, hypoxanthine, hypotaurine, pyridoxamine, pyruvic oxime, pyruvic acid, phenylalanine, phenylpyruvic acid, phenylbutyric acid, psicose, putrescine, proline, pelargonic acid, boric acid, homocysteine, margaric acid, maleic acid, myo-inositol, myristic acid, meso-erythritol, methionine, methionine sulfoxide, monostearin, lactitol, lactose, ribitol, ribulose, ribose, ribonic acid, ribonic acid lactone, malic acid, leucine, benzoic acid, symmetric dimethylarginine, and uric acid in the serum sample; and evaluating quality of the serum sample based on intensity of the molecule acquired by the detection.

A sixth aspect of the present invention relates to an analysis method, which includes: conducting evaluation of a serum sample by the method for evaluating a sample according to the fifth aspect; and conducting analysis of a serum sample based on the evaluation.

A seventh aspect of the present invention relates to a method for detecting a degraded sample, which includes: obtaining a serum sample prepared from human blood; and conducting detection of at least one molecule selected from the group consisting of 1,6-anhydroglucose, 1-hexadecanol, 2-aminooctanoic acid, 2-aminobutyric acid, 2-ketoisovaleric acid, 2-hydroxyglutaric acid, 2-hydroxypyridine, 3-aminoisobutyric acid, 3-aminopropionic acid, β-alanine, 3-indolepropionic acid, 3-sulfinoalanine, 3-hydroxyanthranyl acid, 3-hydroxyisovaleric acid, 3-hydroxypyruvic acid, 3-hydroxypropionic acid, 3-phenyllactic acid, 4-hydroxyphenyllactic acid, 4-hydroxyproline, 5-hydroxymethyl-2-furancarboxylic acid, N6-acetyllysine, N-acetylglutamine, N-acetylserine, S-adenosylhomocysteine, aconitic acid, adipic acid, ascorbic acid, asparagine, aspartic acid, acetylcarnitine, acetylglycine, acetoacetic acid, azelaic acid, adenine, adenosine, adenosine monophosphate, adenosine 3′,5′-cyclic monophosphate, arachidonic acid, alanine, allantoin, argininosuccinic acid, arginine, allose, benzoic acid, isoleucine, inositol, inosine, uracil, uridine, eicosapentaenoic acid, erythrulose, octadecanol, ornithine, oleamide, cadaverine, cabroic acid, galacturonic acid, carnitine, carnosine, xanthine, xylitol, xylulose, xylose, kynurenine, guanosine, guanosine 3′,5′-cyclic monophosphate, glyoxylic acid, glycolic acid, glycine, glycerol-3-phosphate, glucosamine, gluconic acid, glutamic acid, glutaric acid, creatinine, creatine, cholic acid, succinic acid, choline, sarcosine, cystine, cysteine, cytidine, citramalic acid, citrulline, dihydrouracil, dihydroxyacetone phosphate, dimethylglycine, oxalic acid, scyllo-inositol, sucrose, stearic acid, serine, serotonin, sorbitol, sorbose, tyramine, tyrosine, decanoic acid, dopa, dopamine, docosahexaenoic acid, tryptophan, threonine, threonic acid, trehalose, nicotinamide, paraxanthine, valine, pantothenic acid, histidine, asymmetric dimethylarginine, hydroxylamine, hypoxanthine, hypotaurine, pyridoxamine, pyruvic oxime, pyruvic acid, phenylalanine, phenylpyruvic acid, phenylbutyric acid, psicose, putrescine, proline, pelargonic acid, boric acid, homocysteine, margaric acid, maleic acid, myo-inositol, myristic acid, meso-erythritol, methionine, methionine sulfoxide, monostearin, lactitol, lactose, ribitol, ribulose, ribose, ribonic acid, ribonic acid lactone, malic acid, leucine, benzoic acid, symmetric dimethylarginine, and uric acid in the serum sample.

An eighth aspect of the present invention relates to a marker for detecting a degraded serum sample, which contains at least one molecule selected from the group consisting of 1,6-anhydroglucose, 1-hexadecanol, 2-aminooctanoic acid, 2-aminobutyric acid, 2-ketoisovaleric acid, 2-hydroxyglutaric acid, 2-hydroxypyridine, 3-aminoisobutyric acid, 3-aminopropionic acid, β-alanine, 3-indolepropionic acid, 3-sulfinoalanine, 3-hydroxyanthranyl acid, 3-hydroxyisovaleric acid, 3-hydroxypyruvic acid, 3-hydroxypropionic acid, 3-phenyllactic acid, 4-hydroxyphenyllactic acid, 4-hydroxyproline, 5-hydroxymethyl-2-furancarboxylic acid, N6-acetyllysine, N-acetylglutamine, N-acetylserine, S-adenosylhomocysteine, aconitic acid, adipic acid, ascorbic acid, asparagine, aspartic acid, acetylcarnitine, acetylglycine, acetoacetic acid, azelaic acid, adenine, adenosine, adenosine monophosphate, adenosine 3′,5′-cyclic monophosphate, arachidonic acid, alanine, allantoin, argininosuccinic acid, arginine, allose, benzoic acid, isoleucine, inositol, inosine, uracil, uridine, eicosapentaenoic acid, erythrulose, octadecanol, ornithine, oleamide, cadaverine, cabroic acid, galacturonic acid, carnitine, carnosine, xanthine, xylitol, xylulose, xylose, kynurenine, guanosine, guanosine 3′, 5′-cyclic monophosphate, glyoxylic acid, glycolic acid, glycine, glycerol-3-phosphate, glucosamine, gluconic acid, glutamic acid, glutaric acid, creatinine, creatine, cholic acid, succinic acid, choline, sarcosine, cystine, cysteine, cytidine, citramalic acid, citrulline, dihydrouracil, dihydroxyacetone phosphate, dimethylglycine, oxalic acid, scyllo-inositol, sucrose, stearic acid, serine, serotonin, sorbitol, sorbose, tyramine, tyrosine, decanoic acid, dopa, dopamine, docosahexaenoic acid, tryptophan, threonine, threonic acid, trehalose, nicotinamide, paraxanthine, valine, pantothenic acid, histidine, asymmetric dimethylarginine, hydroxylamine, hypoxanthine, hypotaurine, pyridoxamine, pyruvic oxime, pyruvic acid, phenylalanine, phenylpyruvic acid, phenylbutyric acid, psicose, putrescine, proline, pelargonic acid, boric acid, homocysteine, margaric acid, maleic acid, myo-inositol, myristic acid, meso-erythritol, methionine, methionine sulfoxide, monostearin, lactitol, lactose, ribitol, ribulose, ribose, ribonic acid, ribonic acid lactone, malic acid, leucine, benzoic acid, symmetric dimethylarginine, and uric acid.

### Effects of the Invention

According to the present invention, it is possible to accurately evaluate the quality of a blood plasma sample or a serum sample based on a proper marker.

### BRIEF DESCRIPTION OF THE DRAWINGS

FIG. 1 is a flowchart illustrating the flow of an analysis method according to an embodiment.

FIG. 2 is a conceptual diagram for explaining the preparation of a blood plasma sample.

FIG. 3 is a conceptual diagram for explaining the preparation of a serum sample.

### MODE FOR CARRYING OUT THE INVENTION

Hereinafter, embodiments for carrying out the present invention will be described with reference to the drawings. The method for evaluating a sample of the following embodiments is to conduct the detection of a predetermined molecule in a sample derived from human blood and to evaluate the quality of the sample based on the detection.

The predetermined molecule is a molecule of which the concentration in a sample that is poor in quality or is degraded is different from the concentration in a sample that is not poor in quality or is not degraded. Here, “poor in quality” and “degraded” mean that the quantitative values such as concentration corresponding to at least a part of the molecules of analysis targets that are contained in the sample may be changed and it is difficult or impossible to acquire the quantitative values before being changed. The information on the quality of a sample is acquired by detecting the above predetermined molecule in the sample, and thus the above predetermined molecule functions as a marker when evaluating the quality of a sample or for detecting a degraded sample and is hereinafter referred to as a marker.

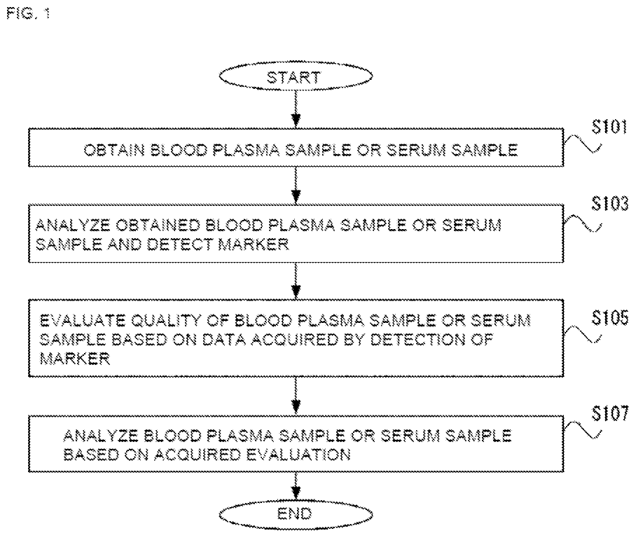

FIG. 1 is a flowchart illustrating the flow of an analysis method including the method for evaluating a sample of the present embodiment. In step S101, a blood plasma sample or a serum sample is obtained. Specific molecules to be detected as markers will be described later.

(Sample)

The sample is not particularly limited as long as it is a blood plasma sample or a serum sample prepared from blood of a human (hereinafter referred to as a blood donor). The blood donor may be a healthy person or a patient suffering from some sort of disease. The method for evaluating a sample of the present embodiment can be applied to samples to be subjected to analysis for arbitrary purposes such as research in addition to analysis for examination or diagnosis of blood donors.

As a suitable example, a case is mentioned in which blood plasma samples or serum samples are prepared and stored at a plurality of facilities and these samples are subjected to analysis at a certain time point in a cohort study. In such a case, when the conditions for preparation or storage are different in different facilities, the results of the analysis vary, and it is difficult to acquire highly reliable results. Hence, by performing the method for evaluating a sample of the present embodiment on at least a part of the stored samples, before the analysis is conducted, to detect a sample that is assumed to be degraded and exclude the sample from the analysis target, the reliability of analysis can be improved.

As described above, the method for evaluating a sample of the present embodiment may be used to evaluate the quality of a blood plasma sample or a serum sample obtained from a patient at a medical institution, a laboratory or the like as well as is used for the research.

When step S101 is ended, step S103 is started. In S103, the blood plasma sample or serum sample obtained in step S101 is analyzed and a marker is detected in vitro. The analysis here is called the first analysis.

(Detection of Marker)

The method for detecting a marker in a blood plasma sample or a serum sample is not particularly limited as long as it is possible to determine with desired accuracy whether or not the concentration of the detected marker satisfies a condition prescribed by a threshold value, a numerical range, or the like.

In the following embodiments, “detecting a marker” refers to performing detection to quantify a marker contained in a blood plasma sample or a serum sample, a substance derived from a marker, such as an ionized marker, a dissociated marker or an ion thereof, or a derivative of a marker or an ion thereof, is directly detected but a case where a marker itself is not directly detected is also included.

From the viewpoint of suitably separating and detecting the intended marker from a sample containing various kinds of substances, the marker contained in a sample is preferably detected by mass spectrometry and is more preferably detected by gas chromatography/mass spectrometry (hereinafter referred to as GC/MS) or liquid chromatography/mass spectrometry (hereinafter referred to as LC/MS). Here, GC/MS and LC/IVIS also include a case of performing multiple times of mass separations such as tandem mass spectrometry and MSⁿ.

Hence, a mass spectrometer is preferable as a detector for detecting a marker contained in a sample in the first analysis. In particular, it is preferable to conduct GC/MS by a gas chromatograph-mass spectrometer (hereinafter referred to as GC-MS) or LC/MS by a liquid chromatograph-mass spectrometer (hereinafter referred to as LC-MS). The mass spectrometer may be a single mass spectrometer or a mass spectrometer capable of conducting two or more mass separation stages. The type of mass analyzer that conducts these mass separations inside the mass spectrometer is also not particularly limited, and the mass analyzer can include one or more of a quadrupole mass filter, an ion trap, a time-of-flight mass analyzer or the like in appropriate combination.

The data acquired by the detection of marker in the first analysis (hereinafter referred to as the first data) is appropriately stored in an arbitrary storage medium. The first data is not particularly limited as long as it indicates the detection signal generated by the detection of marker. The first data can be data corresponding to the mass chromatogram (hereinafter referred to as mass chromatogram data), data corresponding to a mass spectrum (hereinafter, referred to as mass spectrum data) or the like when the first analysis is mass spectrometry. The mass chromatogram data is data indicating the magnitude of the detection signal at each retention time. The mass spectrum data is data indicating the magnitude of the detection signal corresponding to each m/z (corresponding to the mass-to-charge ratio).

When step S103 is ended, step S105 is started. In step S105, the quality of the blood plasma sample or serum sample is evaluated based on the data acquired by the detection of marker in step S103.

In step S105, the marker is quantified by the data analysis of the first data. The calculation in step S105 may be performed manually, but is preferably performed by a control unit or the like including a CPU or the like. The concentration of the marker is calculated using the first data and the calibration data such as the calibration curve or the relative response coefficient acquired in advance. For example, when GC/MS or LC/MS has been conducted in step S103, the peak area or peak intensity of the peak corresponding to the marker in the mass chromatogram is calculated as the magnitude of the detection signal corresponding to the marker (hereinafter, referred to as the detection intensity). The detection intensity is converted using the calibration data to acquire the concentration of the marker in the blood plasma sample or serum sample.

Once the concentration of the marker is acquired, it is determined whether or not the concentration satisfies a prescribed condition (hereinafter referred to as a quality condition) by a threshold value or a numerical range corresponding to the marker. In Tables A to M to be described later, compounds are listed which serve as markers indicating whether or not a predetermined quality condition for preparation or storage of blood plasma samples and serum samples is satisfied. For example, it is assumed that threshold values are prescribed for markers which are listed in Tables A to M and increase under predetermined preparation or storage conditions and the acquired concentrations of the markers are higher than the threshold values. In this case, the sample does not satisfy the quality condition, and the quality of the sample can be evaluated to be insufficient, poor or the like. It is assumed that threshold values are prescribed for markers which are listed in Tables A to M and decrease under predetermined preparation or storage conditions in Tables A to M and the acquired concentrations of the markers are lower than the threshold values. In this case, the sample does not satisfy the quality condition, and the quality of the sample can be evaluated to be insufficient, poor or the like. It is assumed that the numerical ranges are prescribed for markers which are listed in Tables A to M and fluctuate to increase or decrease under predetermined preparation or storage conditions and the concentrations of the markers are out of the numerical ranges. In this case, the sample does not satisfy the quality condition, and the quality of the sample can be evaluated to be insufficient, poor or the like.

In a case where the quality of a sample is evaluated using a plurality of markers, the quality of the sample can be evaluated to be poor when any one or all of the plurality of markers or an arbitrary number of markers do not satisfy the quality condition. In Tables A to M, there are markers of which the detection intensities increase under predetermined conditions as compared with those under the conditions to be comparison target, markers of which the detection intensities decrease, and markers of which the detection intensities fluctuate to increase or decrease, and one or a plurality of these markers can be appropriately combined to evaluate the quality or detect a degraded blood plasma sample or a degraded serum sample. The methods of evaluation algorithm and the like such as the method for setting the quality condition are not particularly limited.

Depending on the marker, it is also possible to evaluate the quality of a sample as being affected by differences in specific conditions in the preparation of a blood plasma sample or serum sample.

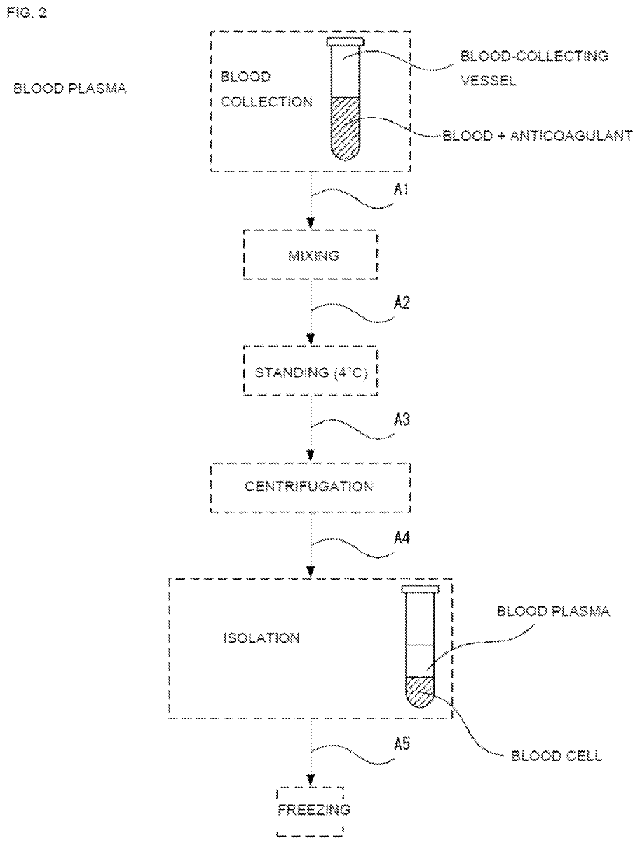

FIG. 2 is a conceptual diagram for explaining the preparation of a blood plasma sample. In the preparation of blood plasma sample, blood is collected from a blood donor to be stored in a blood-collecting vessel or the like containing an anticoagulant such as EDTA. After blood collection, mixing is performed by shaking the blood-collecting vessel containing blood (arrow A1). After mixing, the blood is cooled and allowed to stand at a low temperature such as 4° C. (arrow A2). After standing, the blood is centrifuged (arrow A3). The conditions for centrifugation are not particularly limited as long as the blood plasma is separated, and the centrifugation is performed, for example, at 4° C. and 3000 rpm for 15 minutes. After centrifugation, the blood is separated into blood cells that are precipitates and the supernatant, and the blood plasma contained in the supernatant is isolated (arrow A4). The isolated blood plasma is frozen and stored as a blood plasma sample (arrow A5).

When the marker that is affected by the time from blood collection to centrifugation does not satisfy the quality condition, the blood plasma sample can be evaluated as that the time from blood collection to centrifugation does not satisfy the condition in the preparation of blood plasma sample. Alternatively, the time from blood collection to centrifugation can be estimated by detecting the marker. When the marker that is affected by the time from blood collection to cooling of the blood does not satisfy the quality condition, the blood plasma sample can be evaluated as that the time from blood collection to standing at 4° C. does not satisfy the condition in the preparation of blood plasma sample. Alternatively, the time from blood collection to cooling of the blood can be estimated by detecting the marker. When the marker that is affected by the number of times of freezing and thawing does not satisfy the quality condition, the blood plasma sample can be evaluated as that the number of times of freezing and thawing does not satisfy the condition in the preparation or storage of blood plasma sample. Alternatively, the number of times of freezing and thawing can be estimated by detecting the marker. The information acquired by such quality evaluation can be utilized to match the blood plasma sample used in analysis to certain preparation or storage conditions, and the like.

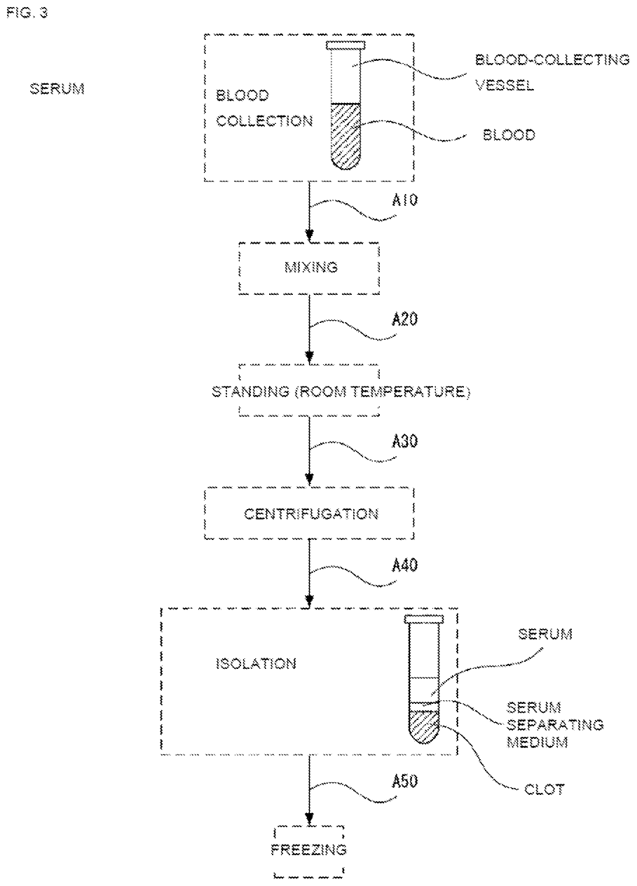

FIG. 3 is a conceptual diagram for explaining the preparation of a serum sample. In the preparation of serum sample, blood is collected from a blood donor to be stored in a blood-collecting vessel or the like that does not contain an anticoagulant. After blood collection, mixing is performed by shaking the blood-collecting vessel containing blood (arrow A10). After mixing, the blood is allowed to stand at room temperature (arrow A20). After standing, the blood is centrifuged (arrow A30). The conditions for centrifugation are not particularly limited as long as the serum is separated, and the centrifugation is performed, for example, at room temperature and 3500 rpm for 5 minutes. After centrifugation, the blood is separated into a clot that is a precipitate, a serum separating medium, and serum, and the serum contained in the supernatant is isolated (arrow A40). The isolated serum is frozen and stored as a serum sample (arrow A50).

When the marker that is affected by the time from blood collection to centrifugation does not satisfy the quality condition, the serum sample can be evaluated as that the time from blood collection to centrifugation does not satisfy the condition in the preparation of serum sample. Alternatively, the time from blood collection to centrifugation can be estimated by detecting the marker. When the marker that is affected by the time from centrifugation to isolation does not satisfy the quality condition, the serum sample can be evaluated as that the time from centrifugation to isolation does not satisfy the condition in the preparation of serum sample. Alternatively, the time from centrifugation to isolation can be estimated by detecting the marker. When the marker that is affected by the number of times of freezing and thawing does not satisfy the quality condition, the serum sample can be evaluated as that the number of times of freezing and thawing does not satisfy the condition in the preparation of serum sample. Alternatively, the number of times of freezing and thawing can be estimated by detecting the marker. The information acquired by such quality evaluation can be utilized to match the serum sample used in analysis to certain preparation or storage conditions, and the like.

Returning to FIG. 1, when step S105 is ended, step S107 is started. Analysis of a blood plasma sample or serum sample is conducted based on the evaluation acquired in step S105. The analysis here is called the second analysis. Based on the evaluation, blood plasma samples or serum samples of poor quality or samples obtained from facilities in which these samples of poor quality have been prepared or stored can be excluded from the analysis target. Alternatively, based on the evaluation, information indicating the reliability of the second analysis may be generated and added to the data acquired by analysis. In the second analysis, the analysis is conducted for arbitrary purposes such as research in addition to analysis for examination or diagnosis of blood donors as described above. It is preferable to conduct the first analysis and the second analysis by the same kind of analysis method from the viewpoint of improving accuracy, but the analysis methods are not particularly limited to this, and the second analysis can be conducted by an arbitrary analysis method. The information acquired by the second analysis is appropriately output to a display unit such as a liquid crystal monitor. When step S107 is ended, the treatment is ended.

(Marker of Blood Plasma Sample)

(1A) In the case of blood plasma samples, the marker can be at least one molecule selected from the group consisting of 1,6-anhydroglucose, 1-hexadecanol, 2-aminobutyric acid, 2-ketobutyric acid, 2′-deoxyuridine, 2-hydroxyisocaproic acid, 2-hydroxypyridine, 3-aminoisobutyric acid, 3-sulfinoalanine, 3-phenyllactic acid, 4-aminobutyric acid, 4-hydroxyphenyllactic acid, 4-hydroxyproline, 5-glutamylcysteine, N6-acetyllysine, N-acetylserine, S-adenosylhomocysteine, S-adenosylmethionine, aconitic acid, ascorbic acid, asparagine, aspartic acid, acetylcarnitine, azelaic acid, adenine, adenosine, adenosine monophosphate, adenosine 3′, 5′-cyclic monophosphate, arachidonic acid, alanine, allantoin, argininosuccinic acid, arginine, isoleucine, inosine, indoxyl sulfate, uridine, octadecanol, ornithine, oleic acid, cabroic acid, galacturonic acid, carnitine, xanthine, xylose, kynurenine, quinolinic acid, guanosine, guanosine monophosphate, glyoxylic acid, glycolic acid, glycine, glycerol-3-phosphate, glutamine, glutamic acid, creatinine, creatine, cholic acid, succinic acid, choline, cholesterol, cystathionine, cystine, cysteine, citicoline, cytidine, cytidine monophosphate, cytosine, citrulline, dihydrouracil, dihydroxyacetone phosphate, dimethylglycine, oxalic acid, scyllo-inositol, sucrose, stearic acid, serotonin, sorbose, symmetric dimethylarginine, dopa, dopamine, docosahexaenoic acid, tryptamine, tryptophan, trehalose, nicotinamide, uric acid, paraxanthine, palmitic acid, pantothenic acid, histamine, histidine, asymmetric dimethylarginine, hydroquinone, hypoxanthine, hypoxanthine, hypotaurine, psicose, proline, boric acid, homocysteine, maleic acid, mannose, myristic acid, methionine sulfoxide, methionine sulfone, monostearin, lactitol, lactose, linoleic acid, ribulose, ribose, ribonic acid, malic acid, leucine, and uric acid.

(1B) When the detection of a marker is conducted by GC/MS of a blood plasma sample, the marker can be at least one molecule selected from the group consisting of 1,6-anhydroglucose, 1-hexadecanol, 2-ketobutyric acid, 2′-deoxyuridine, 2-hydroxyisocaproic acid, 2-hydroxypyridine, 3-aminoisobutyric acid, 3-sulfinoalanine, 3-phenyllactic acid, 4-hydroxyphenyllactic acid, N6-acetyllysine, N-acetylserine, aconitic acid, ascorbic acid, azelaic acid, allantoin, indoxyl sulfate, uridine, octadecanol, oleic acid, cabroic acid, galacturonic acid, xanthine, xylose, quinolinic acid, glyoxylic acid, glycolic acid, glycerol-3-phosphate, creatinine, cholesterol, cytosine, dihydrouracil, dihydroxyacetone phosphate, dimethylglycine, oxalic acid, scyllo-inositol, sucrose, stearic acid, sorbose, docosahexaenoic acid, tryptamine, trehalose, uric acid, paraxanthine, palmitic acid, pantothenic acid, histamine, hydroquinone, hypotaurine, psicose, boric acid, maleic acid, mannose, myristic acid, methionine sulfone, monostearin, lactitol, lactose, linoleic acid, ribulose, ribose, ribonic acid, malic acid, and uric acid.

(1C) When the detection of a marker is conducted by GC/MS of a blood plasma sample, the marker that is affected by the time from when the blood is collected until the blood is subjected to centrifugation can be at least one molecule selected from the group consisting of 1,6-anhydroglucose, 1-hexadecanol, 2-hydroxypyridine, 2-ketobutyric acid, 3-sulfinoalanine, aconitic acid, allantoin, arachidonic acid, ascorbic acid, azelaic acid, cytosine, dihydroxyacetone phosphate, glycerol-3-phosphate, histamine, hydroquinone, lactitol, maleic acid, mannose, methionine sulfone, N-acetylserine, octadecanol, oxalic acid, pantothenic acid, psicose, quinolinic acid, ribonic acid, ribulose, sorbose, sucrose, uridine, xanthine, xylose, docosahexaenoic acid, hypotaurine, trehalose, 2′-deoxyuridine, 3-aminoisobutyric acid, 4-hydroxyphenyllactic acid, cholesterol, dimethylglycine, indoxyl sulfate, lactose, linoleic acid, malic acid, monostearin, myristic acid, oleic acid, palmitic acid, stearic acid, and uric acid.

(1D) When the detection of a marker is conducted by GC/MS of a blood plasma sample, the marker that is affected by the time from when the blood is collected until the blood is subjected to cooling can be at least one molecule selected from the group consisting of 1,6-anhydroglucose, 2′-deoxyuridine, 2-hydroxyisocaproic acid, 2-hydroxypyridine, 2-ketobutyric acid, 3-sulfinoalanine, 3-phenyllactic acid, allantoin, azelaic acid, dihydrouracil, dihydroxyacetone phosphate, docosahexaenoic acid, glycerol-3-phosphate, glycolic acid, glyoxylic acid, histamine, hydroquinone, hypotaurine, lactitol, lactose, maleic acid, mannose, methionine sulfone, N6-acetyllysine, N-acetylserine, oxalic acid, pantothenic acid, paraxanthine, psicose, quinolinic acid, ribose, ribulose, sucrose, trehalose, uric acid, uridine, and xanthine.

(1E) When the detection of a marker is conducted by GC/MS of a blood plasma sample, the marker that is affected by the number of times of freezing and thawing can be at least one molecule selected from the group consisting of 1,6-anhydroglucose, 2-hydroxypyridine, 3-sulfinoalanine, ascorbic acid, azelaic acid, boric acid, cabroic acid, galacturonic acid, hydroquinone, lactose, methionine sulfone, pantothenic acid, psicose, quinolinic acid, ribonic acid, ribulose, sucrose, 2′-deoxyuridine, 2-hydroxyisocaproic acid, cytosine, dihydroxyacetone phosphate, glycerol-3-phosphate, indoxyl sulfate, mannose, monostearin, N6-acetyllysine, N-acetylserine, octadecanol, ribose, scyllo-inositol, trehalose, uridine, xanthine, xylose, 1-hexadecanol (cetanol), 3-phenyllactic acid, allantoin, creatinine, dimethylglycine, histamine, lactitol, maleic acid, and tryptamine.

(1F) When the detection of a marker is conducted by LC/MS of a blood plasma sample, the marker can be at least one molecule selected from the group consisting of 2-aminobutyric acid, 4-aminobutyric acid, 4-hydroxyproline, 5-glutamylcysteine, S-adenosylhomocysteine, S-adenosylmethionine, asparagine, aspartic acid, acetylcarnitine, adenine, adenosine, adenosine monophosphate, adenosine 3′,5′-cyclic monophosphate, alanine, allantoin, argininosuccinic acid, arginine, isoleucine, inosine, uridine, ornithine, carnitine, xanthine, kynurenine, guanosine, guanosine monophosphate, glycine, glutamine, glutamic acid, creatinine, creatine, cholic acid, succinic acid, choline, cystathionine, cystine, cysteine, citicoline, cytidine, cytidine monophosphate, citrulline, dimethylglycine, serotonin, symmetric dimethylarginine, symmetric dimethylarginine, dopa, dopamine, tryptophan, nicotinamide, pantothenic acid, histidine, asymmetric dimethylarginine, hypoxanthine, proline, homocysteine, methionine sulfoxide, malic acid, leucine, and uric acid.

(1G) When the detection of a marker is conducted by LC/MS of a blood plasma sample, the marker that is affected by the time from when the blood is collected until the blood is subjected to centrifugation can be at least one molecule selected from the group consisting of 5-glutamylcysteine, adenosine, adenosine monophosphate, allantoin, citicoline, cysteine, cytidine, cytidine monophosphate, dopa, guanosine monophosphate, hypoxanthine, inosine, nicotinamide, proline, S-adenosylhomocysteine, serotonin, succinic acid, 4-aminobutyric acid, adenine, arginine, aspartic acid, dopamine, guanosine, malic acid, pantothenic acid, S-adenosylmethionine, succinic acid, xanthine, 2-aminobutyric acid, 4-hydroxyproline, acetylcarnitine, adenosine 3′,5′-cyclic monophosphate, alanine, argininosuccinic acid, asymmetric dimethylarginine, carnitine, cholic acid, choline, citrulline, creatine, creatinine, cystathionine, cystine, dimethylglycine, isoleucine, kynurenine, leucine, methionine sulfoxide, symmetric dimethylarginine, tryptophan, uric acid, and uridine.

(1H) When the detection of a marker is conducted by LC/MS of a blood plasma sample, the marker that is affected by the time from when the blood is collected until the blood is subjected to cooling can be at least one molecule selected from the group consisting of 4-aminobutyric acid, 5-glutamylcysteine, adenine, adenosine, adenosine monophosphate, allantoin, aspartic acid, asymmetric dimethylarginine, cholic acid, choline, citicoline, cysteine, cytidine, cytidine monophosphate, dimethylglycine, dopa, dopamine, guanosine monophosphate, hypoxanthine, inosine, nicotinamide, ornithine, proline, S-adenosylhomocysteine, S-adenosylmethionine, serotonin, and xanthine.

(1I) When the detection of a marker is conducted by LC/MS of a blood plasma sample, the marker that is affected by the number of times of freezing and thawing can be at least one molecule selected from the group consisting of 4-aminobutyric acid, 5-glutamylcysteine, adenine, adenosine, adenosine monophosphate, allantoin, arginine, argininosuccinic acid, choline, creatine, creatinine, cystathionine, cysteine, cytidine monophosphate, dopa, malic acid, S-adenosylhomocysteine, S-adenosylmethionine, succinic acid, xanthine, carnitine, citicoline, cytidine, guanosine, guanosine monophosphate, hypoxanthine, inosine, kynurenine, nicotinamide, serotonin, uridine, 4-hydroxyproline, alanine, asparagine, aspartic acid, cholic acid, citrulline, cystine, dimethylglycine, glutamic acid, glutamine, glycine, histidine, homocysteine, isoleucine, leucine, pantothenic acid, and symmetric dimethylarginine.

(Marker of Serum Sample)

(2A) In the case of serum samples, the marker can be at least one molecule selected from the group consisting of 1,6-anhydroglucose, 1-hexadecanol, 2-aminooctanoic acid, 2-aminobutyric acid, 2-ketoisovaleric acid, 2-hydroxyglutaric acid, 2-hydroxypyridine, 3-aminoisobutyric acid, 3-aminopropionic acid (β-alanine), 3-indolepropionic acid, 3-sulfinoalanine, 3-hydroxyanthranyl acid, 3-hydroxyisovaleric acid, 3-hydroxypyruvic acid, 3-hydroxypropionic acid, 3-phenyllactic acid, 4-hydroxyphenyllactic acid, 4-hydroxyproline, 5-hydroxymethyl-2-furancarboxylic acid, N6-acetyllysine, N-acetylglutamine, N-acetylserine, S-adenosylhomocysteine, aconitic acid, adipic acid, ascorbic acid, asparagine, aspartic acid, acetylcarnitine, acetylglycine, acetoacetic acid, azelaic acid, adenine, adenosine, adenosine monophosphate, adenosine 3′,5′-cyclic monophosphate, arachidonic acid, alanine, allantoin, argininosuccinic acid, arginine, allose, benzoic acid, isoleucine, inositol, inosine, uracil, uridine, eicosapentaenoic acid, erythrulose, octadecanol, ornithine, oleamide, cadaverine, cabroic acid, galacturonic acid, carnitine, carnosine, xanthine, xylitol, xylulose, xylose, kynurenine, guanosine, guanosine 3′,5′-cyclic monophosphate, glyoxylic acid, glycolic acid, glycine, glycerol-3-phosphate, glucosamine, gluconic acid, glutamic acid, glutaric acid, creatinine, creatine, cholic acid, succinic acid, choline, sarcosine, cystine, cysteine, cytidine, citramalic acid, citrulline, dihydrouracil, dihydroxyacetone phosphate, dimethylglycine, oxalic acid, scyllo-inositol, sucrose, stearic acid, serine, serotonin, sorbitol, sorbose, tyramine, tyrosine, decanoic acid, dopa, dopamine, docosahexaenoic acid, tryptophan, threonine, threonic acid, trehalose, nicotinamide, paraxanthine, valine, pantothenic acid, histidine, asymmetric dimethylarginine, hydroxylamine, hypoxanthine, hypotaurine, pyridoxamine, pyruvic oxime, pyruvic acid, phenylalanine, phenylpyruvic acid, phenylbutyric acid, psicose, putrescine, proline, pelargonic acid, boric acid, homocysteine, margaric acid, maleic acid, myo-inositol, myristic acid, meso-erythritol, methionine, methionine sulfoxide, monostearin, lactitol, lactose, ribitol, ribulose, ribose, ribonic acid, ribonic acid lactone, malic acid, leucine, benzoic acid, symmetric dimethylarginine, and uric acid.

(2B) When the detection of a marker is conducted by GC/MS of a serum sample, the marker can be at least one molecule selected from the group consisting of 1,6-anhydroglucose, 1-hexadecanol, 2-aminooctanoic acid, 2-aminobutyric acid, 2-ketoisovaleric acid, 2-hydroxyglutaric acid, 2-hydroxypyridine, 3-aminoisobutyric acid, 3-aminopropionic acid, 3-indolepropionic acid, 3-sulfinoalanine, 3-hydroxyanthranylic acid, 3-hydroxyisovaleric acid, 3-hydroxypyruvic acid, 3-hydroxypropionic acid, 3-phenyllactic acid, 4-hydroxyphenyllactic acid, 4-hydroxyproline, 5-hydroxymethyl-2-furancarboxylic acid, N6-acetyllysine, N-acetylglutamine, N-acetylserine, aconitic acid, adipic acid, ascorbic acid, acetylglycine, acetoacetic acid, azelaic acid, adenosine, arachidonic acid, allantoin, arginine, allose, benzoic acid, inositol, uracil, eicosapentaenoic acid, erythrulose, octadecanol, oleamide, cadaverine, cabroic acid, galacturonic acid, xylitol, xylulose, xylose, glyoxylic acid, glycolic acid, glycerol-3-phosphate, glucosamine, gluconic acid, glutaric acid, sarcosine, citramalic acid, dihydrouracil, dihydroxyacetone phosphate, oxalic acid, scyllo-inositol, sucrose, stearic acid, sorbitol, sorbose, tyramine, decanoic acid, dopamine, docosahexaenoic acid, threonic acid, trehalose, paraxanthine, pantothenic acid, hydroxylamine, hypoxanthine, hypotaurine, pyridoxamine, pyruvic oxime, pyruvic acid, phenylpyruvic acid, phenylbutyric acid, psicose, putrescine, pelargonic acid, boric acid, margaric acid, maleic acid, myo-inositol, myristic acid, meso-erythritol, monostearin, lactitol, lactose, ribitol, ribulose, ribose, ribonic acid, ribonic acid lactone, benzoic acid, and uric acid.

(2C) When the detection of a marker is conducted by GC/MS of a serum sample, the marker that is affected by the time from when the blood is collected until the blood is subjected to centrifugation can be at least one molecule selected from the group consisting of 2-aminooctanoic acid, 2-hydroxypyridine, 3-hydroxyanthranyl acid, 3-hydroxypyruvic acid, 3-indolepropionic acid, 3-sulfinoalanine, acetylglycine, aconitic acid, adenosine, adipic acid, allantoin, ascorbic acid, azelaic acid, benzoic acid, cadaverine, citramalic acid, dihydrouracil, dihydroxyacetone phosphate, dopamine, erythrulose, glycerol-3-phosphate, glycolic acid, hypotaurine, hypoxanthine, lactitol, lactose, maleic acid, monostearin, N6-acetyllysine, octadecanol, oxalic acid, pantothenic acid, paraxanthine, pyridoxamine, pyruvic acid, ribose, sorbose, sucrose, tyramine, uracil, xylose, 1,6-anhydroglucose, 2-hydroxyglutaric acid, 2-ketoisovaleric acid, 3-aminopropionic acid, acetoacetic acid, decanoic acid, galacturonic acid, galacturonic acid, glutaric acid, inositol, lactose, meso-erythritol, myo-inositol, myristic acid, psicose, putrescine, ribitol, ribonic acid lactone, ribulose, scyllo-inositol, sorbitol, threonic acid, trehalose, uric acid, xylitol, xylose, xylulose, 1-hexadecanol, 3-hydroxyisovaleric acid, 4-hydroxyproline, dihydrouracil, gluconic acid, N-acetylserine, phenylbutyric acid, and ribonic acid.

(2D) When the detection of a marker is conducted by GC/MS of a serum sample, the marker that is affected by the time from when centrifugation of the blood is conducted until the serum obtained by the centrifugation is isolated can be at least one molecule selected from the group consisting of 1,6-anhydroglucose, 1-hexadecanol, 2-aminooctanoic acid, 2-hydroxyglutaric acid, 2-hydroxypyridine, 3-sulfinoalanine, 4-hydroxyphenyllactic acid, 4-hydroxyproline, 5-hydroxymethyl-2-furancarboxylic acid, aconitic acid, adenosine, adipic acid, azelaic acid, benzoic acid, boric acid, cadaverine, citramalic acid, dihydrouracil, dopamine, erythrulose, galacturonic acid, hypoxanthine, lactitol, lactose, maleic acid, N-acetylserine, octadecanol, pantothenic acid, phenylbutyric acid, psicose, putrescine, pyruvic acid, ribitol, ribonic acid lactone, ribose, sucrose, trehalose, 2-aminobutyric acid, 3-hydroxypropionic acid, 3-hydroxypyruvic acid, 3-indolepropionic acid, acetoacetic acid, allantoin, dihydroxyacetone phosphate, glucosamine, hydroxylamine, lactose, monostearin, N6-acetyllysine, N-acetylglutamine, oxalic acid, paraxanthine, phenylpyruvic acid, pyruvic oxime, threonic acid, tyramine, uracil, and xylulose.

(2E) When the detection of a marker is conducted by GC/MS of a serum sample, the marker that is affected by the number of times of freezing and thawing can be at least one molecule selected from the group consisting of 1,6-anhydroglucose, 2-aminooctanoic acid, 2-hydroxypyridine, 3-hydroxypropionic acid, 3-phenyllactic acid, 3-sulfinoalanine, 4-hydroxyproline, acetoacetic acid, adenosine, boric acid, dihydrouracil, dihydrouracil, dihydroxyacetone phosphate, dopamine, erythrulose, erythrulose, glyoxylic acid, lactose, maleic acid, N6-acetyllysine, oleamide, oxalic acid, pantothenic acid, phenylbutyric acid, psicose, ribonic acid lactone, ribose, threonic acid, 3-hydroxyanthranic acid, allose, cadaverine, lactose, octadecanol, psicose, uracil, 1-hexadecanol, 2-aminobutyric acid, 3-aminoisobutyric acid, 3-hydroxypyruvic acid, 3-indolepropionic acid, adipic acid, allantoin, arachidonic acid, arginine, azelaic acid, benzoic acid, cabroic acid, citramalic acid, docosahexaenoic acid, eicosapentaenoic acid, glucosamine, glycolic acid, hydroxylamine, hypoxanthine, margaric acid, meso-erythritol, monostearin, N-acetylglutamine, pelargonic acid, paraxanthine, phenylpyruvic acid, putrescine, pyridoxamine, pyruvic oxime, ribulose, sarcosine, sorbitol, sorbose, stearic acid, sucrose, trehalose, tyramine, and uric acid.

(2F) When the detection of a marker is conducted by LC/MS of a serum sample, the marker can be at least one molecule selected from the group consisting of 2-aminobutyric acid, 4-hydroxyproline, S-adenosylhomocysteine, asparagine, aspartic acid, acetylcarnitine, adenine, adenosine, adenosine monophosphate, adenosine 3′,5′-cyclic monophosphate, alanine, allantoin, argininosuccinic acid, arginine, isoleucine, inosine, uridine, ornithine, carnitine, carnosine, xanthine, kynurenine, guanosine, guanosine 3′,5′-cyclic monophosphate, glycine, glutamic acid, creatinine, creatine, cholic acid, succinic acid, choline, cystine, cysteine, cytidine, citrulline, dimethylglycine, serine, serotonin, tyrosine, dopa, dopamine, tryptophan, threonine, nicotinamide, valine, pantothenic acid, histidine, asymmetric dimethylarginine, hypoxanthine, phenylalanine, proline, homocysteine, methionine, methionine sulfoxide, malic acid, leucine, symmetric dimethylarginine, and uric acid.

(2G) When the detection of a marker is conducted by LC/MS of a serum sample, the marker that is affected by the time from when the blood is collected until the blood is subjected to centrifugation can be at least one molecule selected from the group consisting of adenosine, adenosine 3′,5′-cyclic monophosphate, allantoin, aspartic acid, carnosine, choline, cytidine, dopa, glutamic acid, guanosine, guanosine 3′,5′-cyclic monophosphate, hypoxanthine, inosine, malic acid, nicotinamide, ornithine, S-adenosylhomocysteine, uridine, xanthine, arginine, argininosuccinic acid, cysteine, methionine sulfoxide, serine, succinic acid, asparagine, proline, histidine, pantothenic acid, isoleucine, leucine, dopamine, and glycine.

(2H) When the detection of a marker is conducted by LC/MS of a serum sample, the marker that is affected by the time from when centrifugation of the blood is conducted until the serum obtained by the centrifugation is isolated can be at least one molecule selected from the group consisting of adenine, adenosine, adenosine monophosphate, argininosuccinic acid, carnosine, cystine, cytidine, glutamic acid, guanosine, guanosine 3′,5′-cyclic monophosphate, inosine, malic acid, S-adenosylhomocysteine, serotonin, adenosine 3′,5′-cyclic monophosphate, allantoin, aspartic acid, cysteine, hypoxanthine, methionine sulfoxide, proline, and xanthine.

(2I) When the detection of a marker is conducted by LC/MS of a serum sample, the marker that is affected by the number of times of freezing and thawing can be at least one molecule selected from the group consisting of adenine, adenosine, adenosine 3′,5′-cyclic monophosphate, adenosine monophosphate, allantoin, carnosine, creatine, cysteine, cystine, cytidine, guanosine 3′,5′-cyclic monophosphate, hypoxanthine, inosine, kynurenine, methionine sulfoxide, succinic acid, uridine, xanthine, 2-aminobutyric acid, 4-hydroxyproline, alanine, arginine, argininosuccinic acid, asparagine, asymmetric dimethylarginine, carnitine, cholic acid, choline, citrulline, creatinine, dimethylglycine, dopa, glycine, guanosine, histidine, homocysteine, isoleucine, leucine, methionine, nicotinamide, S-adenosylhomocysteine, serine, symmetric dimethylarginine, threonine, tryptophan, tyrosine, uric acid, acetylcarnitine, aspartic acid, glutamic acid, malic acid, ornithine, pantothenic acid, phenylalanine, proline, serotonin, and valine.

The markers used to evaluate the quality of blood plasma samples and serum samples can be used as markers for detecting degraded blood plasma samples and markers for detecting degraded serum samples, respectively. A method for detecting a degraded sample is provided, which includes determining that a blood plasma sample or a serum sample is degraded based on the detection of at least one molecule selected from these markers.

(Aspect)

It will be understood by those skilled in the art that the plurality of exemplary embodiments described above are specific examples of the following aspects.

(Paragraph 1) The method for evaluating a sample according to an aspect includes obtaining a blood plasma sample prepared from human blood, conducting detection of at least one molecule presented in (1A) above in the blood plasma sample, and evaluating the quality of the blood plasma sample based on the intensity of the molecule acquired by the detection. This makes it possible to accurately evaluate the quality of a blood plasma sample based on a proper marker.

(Paragraph 2) In the method for evaluating a sample according to another aspect, detection of at least one molecule presented in (1B) above in the blood plasma sample by gas chromatography/mass spectrometry is conducted in the detection in the method for evaluating a sample described in paragraph 1. This makes it possible to accurately evaluate the quality of a blood plasma sample based on a proper marker when detection is conducted by GC/MS.

(Paragraph 3) In the method for evaluating a sample according to another aspect, detection of at least one molecule presented in (1C) above in the blood plasma sample is conducted in the detection and the quality of the blood plasma sample based on the time from when the blood is collected until the blood is subjected to centrifugation is evaluated based on the intensity of the molecule acquired by the detection in the method for evaluating a sample described in paragraph 2. This makes it possible to accurately evaluate the quality of a blood plasma sample relating to this time based on a proper marker when detection is conducted by GC/MS.

(Paragraph 4) In the method for evaluating a sample according to another aspect, detection of at least one molecule presented in (1D) above in the blood plasma sample is conducted in the detection and the quality of the blood plasma sample based on the time from when the blood is collected until the blood is subjected to cooling is evaluated based on the intensity of the molecule acquired by the detection in the method for evaluating a sample described in paragraph 2. This makes it possible to accurately evaluate the quality of a blood plasma sample relating to this time based on a proper marker when detection is conducted by GC/MS.

(Paragraph 5) In the method for evaluating a sample according to another aspect, detection of at least one molecule presented in (1E) above in the blood plasma sample is conducted in the detection and the quality of the blood plasma sample based on the number of times by which the blood plasma sample is subjected to freezing and thawing is evaluated based on the intensity of the molecule acquired by the detection in the method for evaluating a sample described in paragraph 2. This makes it possible to accurately evaluate the quality of a blood plasma sample relating to this number of times based on a proper marker when detection is conducted by GC/MS.

(Paragraph 6) In the method for evaluating a sample according to another aspect, detection of at least one molecule presented in (1F) above in the blood plasma sample by liquid chromatography/mass spectrometry is conducted in the detection in the method for evaluating a sample described in paragraph 1. This makes it possible to accurately evaluate the quality of a blood plasma sample based on a proper marker when detection is conducted by LC/MS.

(Paragraph 7) In the method for evaluating a sample according to another aspect, detection of at least one molecule presented in (1G) above in the blood plasma sample is conducted in the detection and the quality of the blood plasma sample based on the time from when the blood is collected until the blood is subjected to centrifugation is evaluated based on the intensity of the molecule acquired by the detection in the method for evaluating a sample described in paragraph 6. This makes it possible to accurately evaluate the quality of a blood plasma sample relating to this time based on a proper marker when detection is conducted by LC/MS.

(Paragraph 8) In the method for evaluating a sample according to another aspect, detection of at least one molecule presented in (1H) above in the blood plasma sample is conducted in the detection and the quality of the sample based on the time from when the blood is collected until the blood is subjected to cooling is evaluated based on the intensity of the molecule acquired by the detection in the method for evaluating a sample described in paragraph 6. This makes it possible to accurately evaluate the quality of a blood plasma sample relating to this time based on a proper marker when detection is conducted by LC/MS.

(Paragraph 9) In the method for evaluating a sample according to another aspect, detection of at least one molecule presented in (1I) above in the blood plasma sample is conducted in the detection and the quality of the blood plasma sample based on the number of times by which the blood plasma sample is subjected to freezing and thawing is evaluated based on the intensity of the molecule acquired by the detection in the method for evaluating a sample described in paragraph 6. This makes it possible to accurately evaluate the quality of a blood plasma sample relating to this number of times based on a proper marker when detection is conducted by LC/MS.

(Paragraph 10) The analysis method according to another aspect includes conducting evaluation of a blood plasma sample by the method for evaluating a sample described in any one of paragraphs 1 to 9; and conducting analysis of a blood plasma sample based on the evaluation. This makes it possible to match the conditions for preparation of samples and to conduct analysis with high accuracy.

(Paragraph 11) The method for detecting a degraded sample according to another aspect includes obtaining a blood plasma sample prepared from human blood and conducting detection of at least one molecule presented in (1A) above in the blood plasma sample. This makes it possible to accurately evaluate the quality of a blood plasma sample based on a proper marker.

(Paragraph 12) The marker for detecting a degraded blood plasma sample according to another aspect contains at least one molecule presented in (1A) above. This makes it possible to accurately evaluate the quality of a blood plasma sample.

(Paragraph 13) The method for evaluating a sample according to an aspect includes obtaining a serum sample prepared from human blood, conducting detection of at least one molecule presented in (2A) above in the serum sample, and evaluating the quality of the serum sample based on the intensity of the molecule acquired by the detection. This makes it possible to accurately evaluate the quality of a serum sample based on a proper marker.

(Paragraph 14) In the method for evaluating a sample according to another aspect, detection of at least one molecule presented in (2B) above in the serum sample by gas chromatography/mass spectrometry is conducted in the detection in the method for evaluating a sample described in paragraph 13. This makes it possible to accurately evaluate the quality of a serum sample based on a proper marker when detection is conducted by GC/MS.

(Paragraph 15) In the method for evaluating a sample according to another aspect, detection of at least one molecule presented in (2C) above in the serum sample is conducted in the detection and the quality of the serum sample based on the time from when the blood is collected until the blood is subjected to centrifugation is evaluated based on the intensity of the molecule acquired by the detection in the method for evaluating a sample described in paragraph 14. This makes it possible to accurately evaluate the quality of a serum sample relating to this time based on a proper marker when detection is conducted by GC/MS.

(Paragraph 16) In the method for evaluating a sample according to another aspect, detection of at least one molecule presented in (2D) above in the serum sample is conducted in the detection and the quality of the sample based on the time from when centrifugation of the blood is conducted until the serum obtained by the centrifugation is isolated is evaluated based on the intensity of the molecule acquired by the detection in the method for evaluating a sample described in paragraph 14. This makes it possible to accurately evaluate the quality of a serum sample relating to this time based on a proper marker when detection is conducted by GC/MS.

(Paragraph 17) In the method for evaluating a sample according to another aspect, detection of at least one molecule presented in (2E) above in the serum sample is conducted in the detection and the quality of the serum sample based on the number of times by which the serum sample is subjected to freezing and thawing is evaluated based on the intensity of the molecule acquired by the detection in the method for evaluating a sample described in paragraph 14. This makes it possible to accurately evaluate the quality of a serum sample relating to this number of times based on a proper marker when detection is conducted by GC/MS.

(Paragraph 18) In the method for evaluating a sample according to another aspect, detection of at least one molecule presented in (2F) above in the serum sample by liquid chromatography/mass spectrometry is conducted in the detection in the method for evaluating a sample described in paragraph 13. This makes it possible to accurately evaluate the quality of a serum sample based on a proper marker when detection is conducted by LC/MS.

(Paragraph 19) In the method for evaluating a sample according to another aspect, detection of at least one molecule presented in (2G) above in the serum sample is conducted in the detection and the quality of the serum sample based on the time from when the blood is collected until the blood is subjected to centrifugation is evaluated based on the intensity of the molecule acquired by the detection in the method for evaluating a sample described in paragraph 18. This makes it possible to accurately evaluate the quality of a serum sample changed relating to this time based on a proper marker when detection is conducted by LC/MS.

(Paragraph 20) In the method for evaluating a sample according to another aspect, detection of at least one molecule presented in (2H) above in the serum sample is conducted in the detection and the quality of the serum sample based on the time from when centrifugation of the blood is conducted until the serum obtained by the centrifugation is isolated is evaluated based on the intensity of the molecule acquired by the detection in the method for evaluating a sample described in paragraph 18. This makes it possible to accurately evaluate the quality of a serum sample changed relating to this time based on a proper marker when detection is conducted by LC/MS.

(Paragraph 21) In the method for evaluating a sample according to another aspect, detection of at least one molecule presented in (2I) above in the serum sample is conducted in the detection and the quality of the serum sample based on the number of times by which the serum sample is subjected to freezing and thawing is evaluated based on the intensity of the molecule acquired by the detection in the method for evaluating a sample described in paragraph 18. This makes it possible to accurately evaluate the quality of a serum sample relating to this number of times based on a proper marker when detection is conducted by LC/MS.

(Paragraph 22) The analysis method according to another aspect includes conducting evaluation of a serum sample by the method for evaluating a sample described in any one of paragraphs 13 to 21; and conducting analysis of a serum sample based on the evaluation. This makes it possible to match the conditions for preparation of samples and to conduct analysis with high accuracy.

(Paragraph 23) The method for detecting a degraded sample according to another aspect includes obtaining a serum sample prepared from human blood and conducting detection of at least one molecule presented in (2A) above in the serum sample. This makes it possible to accurately evaluate the quality of a serum sample based on a proper marker.

(Paragraph 24) The marker for detecting a degraded serum sample according to another aspect contains at least one molecule presented in (2A) above. This makes it possible to accurately evaluate the quality of a serum sample.

The present invention is not limited to the contents of the above embodiments. Other aspects considered within the scope of the technical idea of the present invention are also included within the scope of the present invention.

### Examples

Hereinafter, Examples according to the above-described embodiments will be described, but the present invention is not limited to the specific instruments, conditions or the like in the following Examples.

Interviews were conducted with facilities in which blood was collected and blood plasma samples and serum samples were stored, and information on the permissible range such as the time from blood collection to centrifugation of blood in the preparation of blood plasma samples and serum samples was acquired. Based on this information, blood plasma samples and serum samples were prepared under a plurality of different conditions.

Preparation of Blood Plasma Sample

At room temperature, 5 mL of blood from a healthy subject was taken into a blood-collecting vessel containing EDTA, and the blood-collecting vessel was inverted for mixing, then cooled, and allowed to stand at 4° C. Here, in order to investigate how the detection intensity of molecules contained in the sample was affected in a case where the time to cooling was set to 5 minutes or more as compared to that in a case where the time to cooling was set to 1 minute or less, preparations were performed under both the former condition and the latter condition. After standing, the blood sample was subjected to centrifugation under a condition of 4° C., 3000 rpm, and 15 minutes. Here, in order to investigate how the detection intensity of molecules contained in the sample was affected in cases where the time from blood collection to centrifugation was set to 1 hour, 4 hours, 8 hours, and 12 hours as compared to that in a case where the time was set to 15 minutes, preparations were performed under the respective conditions of 15 minutes, 1 hour, 4 hours, 8 hours, and 12 hours. After centrifugation, the blood-collecting vessel was left to stand at room temperature for 30 minutes, and the blood plasma was isolated during this 30 minutes. The obtained blood plasma sample was frozen and stored. Here, in order to investigate how the detection intensity of molecules contained in the sample was affected in cases where freezing and thawing was performed 4 times, 6 times, and 10 times after freezing as compared to that in a case where freezing and thawing was performed 2 times after freezing, freezing and thawing was performed under the respective conditions of 2, 4, 6, and 10 times.

Preparation of Serum Sample

At room temperature, 4 mL of blood from a healthy subject was taken into a blood-collecting vessel that did not contain an anticoagulant, and the blood-collecting vessel was inverted for mixing, and then allowed to stand at room temperature. After standing, the blood sample was subjected to centrifugation under a condition of room temperature, 3500 rpm, and 5 minutes. Here, in order to investigate how the detection intensity of molecules contained in the sample was affected in cases where the time from blood collection to centrifugation was set to 1 hour, 4 hours, 8 hours, and 12 hours as compared to that in a case where the time was set to 15 minutes, preparations were performed under the respective conditions of 15 minutes, 1 hour, 4 hours, 8 hours, and 12 hours. After centrifugation, the blood-collecting vessel was left to stand at room temperature, and the serum was isolated during this time. Here, in order to investigate how the detection intensity of molecules contained in the sample was affected in cases where the time during which the blood-collecting vessel was left at room temperature was set to 1 hour and 6 hours as compared to that in a case where the time was set to 30 minutes, preparations were performed under the respective conditions of 30 minutes, 1 hour, and 6 hours. The obtained serum sample was frozen and stored. Here, in order to investigate how the detection intensity of molecules contained in the sample was affected in cases where freezing and thawing was performed 4 times, 6 times, and 10 times after freezing as compared to that in a case where freezing and thawing was performed 2 times after freezing, freezing and thawing was performed under the respective conditions of 2, 4, 6, and 10 times.

Analysis

Frozen blood plasma samples and serum samples were subjected to GC/MS or LC/MS. In GC/MS, the blood plasma samples and serum samples were subjected to methoxymation and trimethylsilylation and then introduced to GC-MS.

GC/MS

GC/MS was conducted using GCMS·TQ8040 (Shimadzu Corporation) that was a GC·MS equipped with AOC·20i (Shimadzu Corporation) as an autosampler.

Condition for Gas Chromatography

Number and order of cleaning before injection: 3 times

(cleaning with acetone 2 times and then with pyridine 1 time)

Number and order of cleaning after injection: 7 times

(cleaning with acetone 5 times and then with pyridine 2 times)

Column: BPX5 (inner diameter of 0.25 mm, length of 30 m, and film thickness of 0.25 μm) (SGE)

Column temperature: maintained at 60° C. for 2 minutes, then raised at 15° C./min, and maintained at 330° C. for 3 minutes.

Inlet temperature: 250° C.

Carrier gas: helium

Carrier gas control mode: constant linear velocity of 39.0 cm/sec

Sample introduction method: split (split ratio of 30:1) Injection volume: 1 μL

Condition for Mass Spectrometry

Ionization method: electron ionization

Ionization voltage: 70 V

Ionization current: 60 μA

Interface temperature: 280° C.

Ion source temperature: 200° C.

Gain: reference value (relative auto tuning result value of +0.35 kV)

Mode: multiple reaction monitoring (MRM)

LC/MS

LC/MS was conducted using LCMS-8050 (Shimadzu Corporation) that was a triple quadrupole LC·MS.

Condition for Liquid Chromatography

Analysis column: Discovery HS F5-3 (inner diameter of 2.1 mm, length of 150 mm, film thickness of 3 μm) (Sigma-Aldrich)

Column temperature: 40° C.

Injection volume: 3 μL

Mobile phase:

(A) 0.1% formic acid (dissolved in water)

(B) 0.1% formic acid (dissolved in acetonitrile)

Flow velocity: 0.25 mL/min

Gradient program:

| time (minutes) | concentration of mobile phase B (%) |
|---|---|
| 0 | 0 |
| 2.0 | 0 |
| 5.0 | 25 |
| 11.0 | 35 |
| 15.0 | 95 |
| 20.0 | 95 |
| 20.1 | 0 |
| 25.0 | stop |

Condition for Mass Spectrometry

Ionization method: electrospray

Temperature:
Desolvation Line (DL) temperature: 250° C.
Heat block temperature: 400° C.
Interface temperature: 300° C.

Gas flow rate:
Nebulizer gas flow rate: 3.0 L/min
Drying gas flow rate: 10.0 L/min
Heating gas flow rate: 10.0 L/min

Mode: multiple reaction monitoring (MRM)

Result

Table A presents compounds of which the detection intensity increased or decreased by 30% or more in cases where the time from blood collection to centrifugation, when a blood plasma sample was analyzed by GC/MS, was set to 1 hour, 4 hours, 8 hours, and 12 hours as compared to that in a case where the time was set to 15 minutes. The rate of increase or decrease in Table A is the value of detection intensity relative to 1 that is the detection intensity in a case where the time from blood collection to centrifugation is set to 15 minutes.

TABLE A
compounds affected by time from blood collection
to centrifugation in GC/MS of blood plasma sample

| | Time when compound | | Rate of increase |
|---|---|---|---|
| Compound name | is affected | Increase or decrease | or decrease |
| 1,6-Anhydroglucose | After 4 hours | Increase | 1.42 |
| 1-Hexadecanol (cetanol) | After 4 hours | Decrease | 0.60 |
| 2-Hydroxypyridine | After 4 hours | Decrease | 0.57 |
| 2-Ketobutyric acid | After 4 hours | Different from individual | 1.48/0.64 |
| | | to individual | |
| 3-Sulfinoalanine | After 4 hours | Different from individual | 1.88/0.32 |
| | | to individual | |
| Aconitic acid | After 4 hours | Decrease | 0.41 |
| Allantoin | After 4 hours | Decrease | 0.18 |
| Arachidonic acid | After 4 hours | Increase | 1.56 |
| Ascorbic acid | After 4 hours | Increase | 1.77 |
| Azelaic acid | After 4 hours | Increase | 2.03 |
| Cytosine | After 4 hours | Increase | 1.50 |
| Dihydroxyacetone | After 4 hours | Different from individual | 3.64/0.38 |
| phosphate | | to individual | |
| Glycerol-3-phosphate | After 4 hours | Different from individual | 4.90/0.32 |
| | | to individual | |
| Histamine | After 4 hours | Increase | 1.78 |
| Hydroquinone | After 4 hours | Increase | 17.48 |
| Lactitol | After 4 hours | Different from individual | 3.36/0.22 |
| | | to individual | |
| Maleic acid | After 4 hours | Different from individual | 1.75/0.47 |
| | | to individual | |
| Mannose | After 4 hours | Different from individual | 4.61/0.47 |
| | | to individual | |
| Methionine sulfone | After 4 hours | Increase | 4.98 |
| N-acetylserine | After 4 hours | Different from individual | 1.69/0.66 |
| | | to individual | |
| Octadecanol | After 4 hours | Increase | 1.62 |
| Oxalic acid | After 4 hours | Different from individual | 3.80/0.32 |
| | | to individual | |
| Pantothenic acid | After 4 hours | Increase | 3.15 |
| Psicose | After 4 hours | Increase | 4.33 |
| Quinolinic acid | After 4 hours | Increase | 2.24 |
| Ribonic acid | After 4 hours | Decrease | 0.17 |
| Ribulose | After 4 hours | Different from individual | 4.41/0.66 |
| | | to individual | |
| Sorbose | After 4 hours | Different from individual | 2.10/0.67 |
| | | to individual | |
| Sucrose | After 4 hours | Different from individual | 2.53/0.69 |
| | | to individual | |
| Uridine | After 4 hours | Different from individual | 1.48/0.48 |
| | | to individual | |
| Xanthine | After 4 hours | Different from individual | 1.83/0.49 |
| | | to individual | |
| Xylose | After 4 hours | Different from individual | 1.68/0.69 |
| | | to individual | |
| Docosahexaenoic acid | After 8 hours | Increase | 1.48 |
| Hypotaurine | After 8 hours | Increase | 1.36 |
| Trehalose | After 8 hours | Decrease | 0.56 |
| 2′-Deoxyuridine | After 12 hours | Increase | 1.43 |
| 3-Aminoisobutyric acid | After 12 hours | Increase | 1.44 |
| 4-Hydroxyphenyllactic acid | After 12 hours | Increase | 1.33 |
| Cholesterol | After 12 hours | Increase | 1.38 |
| Dimethylglycine | After 12 hours | Increase | 1.41 |
| Indoxyl sulfate | After 12 hours | Increase | 1.50 |
| Lactose | After 12 hours | Decrease | 0.70 |
| Linoleic acid | After 12 hours | Increase | 1.34 |
| Malic acid | After 12 hours | Increase | 1.36 |
| Monostearin | After 12 hours | Increase | 1.35 |
| Myristic acid | After 12 hours | Increase | 1.55 |
| Oleic acid | After 12 hours | Increase | 4.05 |
| Palmitic acid | After 12 hours | Increase | 4.05 |
| Stearic acid | After 12 hours | Increase | 2.13 |
| Uric acid | After 12 hours | Increase | 1.89 |

Table B presents compounds of which the detection intensity increased or decreased by 30% or more in a case where the time from blood collection to cooling of blood, when a blood plasma sample was analyzed by GC/MS, was set to 5 minutes or more as compared to that in a case where the time was set to 1 minute or less. The rate of increase or decrease in Table B is the value of detection intensity relative to 1 that is the detection intensity in a case where the time from blood collection to cooling is set to 1 minute or less.

TABLE B
compounds affected by time from blood collection to
cooling of blood in GC/MS of blood plasma sample

| | Time when compound | | Rate of increase |
|---|---|---|---|
| Compound name | is affected | Increase or decrease | or decrease |
| 1,6-Anhydroglucose | 5 minutes | Decrease | 0.62 |
| 2′-Deoxyuridine | 5 minutes | Decrease | 0.68 |
| 2-Hydroxyisocaproic acid | 5 minutes | Increase | 1.31 |
| 2-Hydroxypyridine | 5 minutes | Decrease | 0.59 |
| 2-Ketobutyric acid | 5 minutes | Decrease | 0.67 |
| 3-Sulfinoalanine | 5 minutes | Decrease | 0.61 |
| 3-Phenyllactic acid | 5 minutes | Different from individual | 1.47/0.60 |
| | | to individual | |
| Allantoin | 5 minutes | Different from individual | 1.30/0.57 |
| | | to individual | |
| Azelaic acid | 5 minutes | Decrease | 0.67 |
| Dihydrouracil | 5 minutes | Increase | 1.42 |
| Dihydroxyacetone | 5 minutes | Decrease | 0.62 |
| phosphate | | | |
| Docosahexaenoic acid | 5 minutes | Increase | 1.38 |
| Glycerol-3-phosphate | 5 minutes | Increase | 1.49 |
| Glycolic acid | 5 minutes | Decrease | 0.61 |
| Glyoxylic acid | 5 minutes | Increase | 1.37 |
| Histamine | 5 minutes | Decrease | 0.67 |
| Hydroquinone | 5 minutes | Decrease | 0.66 |
| Hypotaurine | 5 minutes | Increase | 1.43 |
| Lactitol | 5 minutes | Decrease | 0.67 |
| Lactose | 5 minutes | Decrease | 0.56 |
| Maleic acid | 5 minutes | Different from individual | 1.38/0.54 |
| | | to individual | |
| Mannose | 5 minutes | Decrease | 0.64 |
| Methionine sulfone | 5 minutes | Decrease | 0.70 |
| N6-acetyllysine | 5 minutes | Decrease | 0.61 |
| N-acetylserine | 5 minutes | Different from individual | 1.64/0.69 |
| | | to individual | |
| Oxalic acid | 5 minutes | Increase | 1.58 |
| Pantothenic acid | 5 minutes | Increase | 3.56 |
| Paraxanthine | 5 minutes | Different from individual | 2.33/0.67 |
| | | to individual | |
| Psicose | 5 minutes | Different from individual | 3.24/0.43 |
| | | to individual | |
| Psicose | 5 minutes | Different from individual | 1.94/0.27 |
| | | to individual | |
| Quinolinic acid | 5 minutes | Different from individual | 1.67/0.41 |
| | | to individual | |
| Ribose | 5 minutes | Increase | 2.09 |
| Ribulose | 5 minutes | Increase | 1.96 |
| Sucrose | 5 minutes | Different from individual | 2.20/0.69 |
| | | to individual | |
| Trehalose | 5 minutes | Different from individual | 5.01/0.58 |
| | | to individual | |
| Uric acid | 5 minutes | Decrease | 0.49 |
| Uridine | 5 minutes | Different from individual | 2.62/0.64 |
| | | to individual | |
| Xanthine | 5 minutes | Decrease | 0.45 |

Table C presents compounds of which the detection intensity increased or decreased by 30% or more in cases where the number of times of freezing and thawing was set to 4 times, 6 times, and 10 times as compared to that in a case where the number of times was set to 2 times when a blood plasma sample was analyzed by GC/MS. The rate of increase or decrease in Table C is the value of detection intensity relative to 1 that is the detection intensity in a case where the number of times of freezing and thawing is set to 2 times.

TABLE C
compounds affected by number of times of freezing
and thawing in GC/MS of blood plasma sample

| | Number of times when | | Rate of increase |
|---|---|---|---|
| Compound name | compound is affected | Increase or decrease | or decrease |
| 1,6-Anhydroglucose | 4 times | Different from individual | 3.49/0.42 |
| | | to individual | |
| 2-Hydroxypyridine | 4 times | Different from individual | 2.48/0.54 |
| | | to individual | |
| 3-Sulfinoalanine | 4 times | Different from individual | 1.59/0.58 |
| | | to individual | |
| Ascorbic acid | 4 times | Increase | 1.41 |
| Azelaic acid | 4 times | Different from individual | 1.45/0.66 |
| | | to individual | |
| Boric acid | 4 times | Increase | 1.56 |
| Cabroic acid | 4 times | Increase | 1.58 |
| Galacturonic acid | 4 times | Increase | 1.44 |
| Hydroquinone | 4 times | Different from individual | 3.54/0.69 |
| | | to individual | |
| Lactose | 4 times | Increase | 1.40 |
| Methionine sulfone | 4 times | Increase | 1.77 |
| Pantothenic acid | 4 times | Increase | 3.07 |
| Psicose | 4 times | Different from individual | 1.77/0.70 |
| | | to individual | |
| Quinolinic acid | 4 times | Different from individual | 1.70/0.68 |
| | | to individual | |
| Ribonic acid | 4 times | Increase | 1.62 |
| Ribulose | 4 times | Increase | 1.38 |
| Sucrose | 4 times | Decrease | 0.53 |
| 2′-Deoxyuridine | 6 times | Different from individual | 1.43/0.67 |
| | | to individual | |
| 2-Hydroxyisocaproic acid | 6 times | Decrease | 0.66 |
| Cytosine | 6 times | Different from individual | 1.93/0.62 |
| | | to individual | |
| Dihydroxyacetone | 6 times | Decrease | 0.42 |
| phosphate | | | |
| Glycerol-3-phosphate | 6 times | Decrease | 0.41 |
| Indoxyl sulfate | 6 times | Decrease | 0.68 |
| Mannose | 6 times | Decrease | 0.51 |
| Monostearin | 6 times | Decrease | 0.67 |
| N6-acetyllysine | 6 times | Decrease | 0.57 |
| N-acetylserine | 6 times | Different from individual | 1.39/0.69 |
| | | to individual | |
| Octadecanol | 6 times | Different from individual | 1.63/0.68 |
| | | to individual | |
| Ribose | 6 times | Different from individual | 1.39/0.70 |
| | | to individual | |
| Scyllo-inositol | 6 times | Decrease | 0.68 |
| Trehalose | 6 times | Increase | 1.98 |
| Uridine | 6 times | Decrease | 0.69 |
| Xanthine | 6 times | Different from individual | 2.21/0.51 |
| | | to individual | |
| Xylose | 6 times | Decrease | 0.68 |
| 1-Hexadecanol (cetanol) | 10 times | Increase | 1.88 |
| 3-Phenyllactic acid | 10 times | Decrease | 0.62 |
| Allantoin | 10 times | Increase | 1.42 |
| Creatinine | 10 times | Increase | 1.43 |
| Dimethylglycine | 10 times | Increase | 1.34 |
| Histamine | 10 times | Increase | 1.63 |
| Lactitol | 10 times | Increase | 1.73 |
| Maleic acid | 10 times | Increase | 2.12 |
| Tryptamine | 10 times | Decrease | 0.69 |

Table D presents compounds of which the detection intensity increased or decreased by 30% or more in cases where the time from blood collection to centrifugation when a blood plasma sample was analyzed by LC/MS was set to 1 hour, 4 hours, 8 hours, and 12 hours as compared to that in a case where the time was set to 15 minutes. The rate of increase or decrease in Table D is the value of detection intensity relative to 1 that is the detection intensity in a case where the time from blood collection to centrifugation is set to 15 minutes.

TABLE D
compounds affected by time from blood collection
to centrifugation in LC/MS of blood plasma sample

| | Time when compound | | Rate of increase |
|---|---|---|---|
| Compound name | is affected | Increase or decrease | or decrease |
| 5-Glutamylcysteine | After 4 hours | Different from individual | 1.79/0.70 |
| | | to individual | |
| Adenosine | After 4 hours | Increase | 1.39 |
| Adenosine monophosphate | After 4 hours | Increase | 1.56 |
| Allantoin | After 4 hours | Decrease | 0.52 |
| Citicoline | After 4 hours | Increase | 1.55 |
| Cysteine | After 4 hours | Increase | 1.33 |
| Cytidine | After 4 hours | Increase | 1.95 |
| Cytidine monophosphate | After 4 hours | Increase | 1.75 |
| Dopa | After 4 hours | Increase | 2.57 |
| Guanosine monophosphate | After 4 hours | Increase | 1.89 |
| Hypoxanthine | After 4 hours | Different from individual | 3.84/0.70 |
| | | to individual | |
| Inosine | After 4 hours | Increase | 2.26 |
| Nicotinamide | After 4 hours | Increase | 1.44 |
| Proline | After 4 hours | Increase | 2.20 |
| S-adenosylhomocysteine | After 4 hours | Decrease | 0.66 |
| Serotonin | After 4 hours | Increase | 2.95 |
| Succinic acid | After 4 hours | Decrease | 0.50 |
| 4-Aminobutyric acid | After 6 hours | Decrease | 0.70 |
| Adenine | After 6 hours | Decrease | 0.63 |
| Arginine | After 6 hours | Decrease | 0.69 |
| Aspartic acid | After 6 hours | Increase | 1.46 |
| Dopamine | After 6 hours | Decrease | 0.53 |
| Guanosine | After 6 hours | Increase | 1.57 |
| Malic acid | After 6 hours | Decrease | 0.58 |
| Pantothenic acid | After 6 hours | Increase | 1.32 |
| S-adenosylmethionine | After 6 hours | Decrease | 0.62 |
| Succinic acid | After 6 hours | Decrease | 0.61 |
| Xanthine | After 6 hours | Increase | 1.39 |
| 2-Aminobutyric acid | After 12 hours | Different from individual | 1.53/0.65 |
| | | to individual | |
| 4-Hydroxyproline | After 12 hours | Different from individual | 1.33/0.69 |
| | | to individual | |
| Acetylcarnitine | After 12 hours | Different from individual | 1.69/0.64 |
| | | to individual | |
| Adenosine 3′,5′-cyclic | After 12 hours | Different from individual | 1.49/0.65 |
| monophosphate | | to individual | |
| Alanine | After 12 hours | Different from individual | 1.41/0.64 |
| | | to individual | |
| Argininosuccinic acid | After 12 hours | Decrease | 0.61 |
| Asymmetric | After 12 hours | Different from individual | 1.78/0.66 |
| dimethylarginine | | to individual | |
| Carnitine | After 12 hours | Different from individual | 1.57/0.64 |
| | | to individual | |
| Cholic acid | After 12 hours | Increase | 1.99 |
| Choline | After 12 hours | Different from individual | 1.83/0.70 |
| | | to individual | |
| Citrulline | After 12 hours | Decrease | 0.68 |
| Creatine | After 12 hours | Different from individual | 1.67/0.66 |
| | | to individual | |
| Creatinine | After 12 hours | Different from individual | 1.59/0.65 |
| | | to individual | |
| Cystathionine | After 12 hours | Increase | 1.44 |
| Cystine | After 12 hours | Decrease | 0.45 |
| Dimethylglycine | After 12 hours | Different from individual | 1.57/0.63 |
| | | to individual | |
| Isoleucine | After 12 hours | Different from individual | 1.31/0.65 |
| | | to individual | |
| Kynurenine | After 12 hours | Different from individual | 1.85/0.66 |
| | | to individual | |
| Leucine | After 12 hours | Decrease | 0.69 |
| Methionine sulfoxide | After 12 hours | Different from individual | 1.36/0.54 |
| | | to individual | |
| Symmetric | After 12 hours | Different from individual | 1.78/0.68 |
| dimethylarginine | | to individual | |
| Tryptophan | After 12 hours | Different from individual | 1.62/0.66 |
| | | to individual | |
| Uric acid | After 12 hours | Increase | 1.52 |
| Uridine | After 12 hours | Different from individual | 1.67/0.60 |
| | | to individual | |

Table E presents compounds of which the detection intensity increased or decreased by 30% or more in a case where the time from blood collection to cooling of blood, when a blood plasma sample was analyzed by LC/MS, was set to 5 minutes or more as compared to that in a case where the time was set to 1 minute or less. The rate of increase or decrease in Table E is the value of detection intensity relative to 1 that is the detection intensity in a case where the time from blood collection to cooling is set to 1 minute or less.

TABLE E
compounds affected by time from blood collection to
cooling of blood in LC/MS of blood plasma sample

| | Time when compound | | Rate of increase |
|---|---|---|---|
| Compound name | is affected | Increase or decrease | or decrease |
| 4-Aminobutyric acid | 5 minutes | Increase | 1.50 |
| 5-Glutamylcysteine | 5 minutes | Increase | 1.33 |
| Adenine | 5 minutes | Increase | 1.31 |
| Adenosine | 5 minutes | Increase | 1.52 |
| Adenosine monophosphate | 5 minutes | Different from individual | 1.43/0.62 |
| | | to individual | |
| Allantoin | 5 minutes | Different from individual | 1.57/0.40 |
| | | to individual | |
| Aspartic acid | 5 minutes | Increase | 1.93 |
| Asymmetric | 5 minutes | Increase | 1.31 |
| dimethylarginine | | | |
| Cholic acid | 5 minutes | Increase | 1.36 |
| Choline | 5 minutes | Increase | 1.37 |
| Citicoline | 5 minutes | Different from individual | 1.48/0.38 |
| | | to individual | |
| Cysteine | 5 minutes | Increase | 1.67 |
| Cytidine | 5 minutes | Different from individual | 2.56/0.46 |
| | | to individual | |
| Cytidine monophosphate | 5 minutes | Increase | 1.34 |
| Dimethylglycine | 5 minutes | Decrease | 0.66 |
| Dopa | 5 minutes | Increase | 2.30 |
| Dopamine | 5 minutes | Increase | 1.43 |
| Guanosine monophosphate | 5 minutes | Different from individual | 1.47/0.59 |
| | | to individual | |
| Hypoxanthine | 5 minutes | Increase | 7.92 |
| Inosine | 5 minutes | Different from individual | 18.25/0.64 |
| | | to individual | |
| Nicotinamide | 5 minutes | Increase | 1.40 |
| Ornithine | 5 minutes | Increase | 1.32 |
| Proline | 5 minutes | Increase | 1.41 |
| S-adenosylhomocysteine | 5 minutes | Different from individual | 1.40/0.49 |
| | | to individual | |
| S-adenosylmethionine | 5 minutes | Increase | 1.60 |
| Serotonin | 5 minutes | Different from individual | 1.62/0.63 |
| | | to individual | |
| Xanthine | 5 minutes | Increase | 1.47 |

Table F presents compounds of which the detection intensity increased or decreased by 30% or more in cases where the number of times of freezing and thawing, when a blood plasma sample was analyzed by LC/MS, was set to 4 times, 6 times, and 10 times as compared to that in a case where the number of times was set to 2 times. The rate of increase or decrease in Table F is the value of detection intensity relative to 1 that is the detection intensity in a case where the number of times of freezing and thawing is set to 2 times.

TABLE F
compounds affected by number of times of freezing
and thawing in LC/MS of blood plasma sample

| | Number of times when | | Rate of increase |
|---|---|---|---|
| Compound name | compound is affected | Increase or decrease | or decrease |
| 4-Aminobutyric acid | 4 times | Increase | 1.76 |
| 5-Glutamylcysteine | 4 times | Increase | 1.32 |
| Adenine | 4 times | Increase | 1.37 |
| Adenosine | 4 times | Decrease | 0.58 |
| Adenosine monophosphate | 4 times | Increase | 1.50 |
| Allantoin | 4 times | Decrease | 0.55 |
| Arginine | 4 times | Increase | 1.34 |
| Argininosuccinic acid | 4 times | Increase | 2.01 |
| Choline | 4 times | Increase | 1.35 |
| Creatine | 4 times | Increase | 1.39 |
| Creatinine | 4 times | Increase | 1.31 |
| Cystathionine | 4 times | Increase | 1.52 |
| Cysteine | 4 times | Increase | 1.99 |
| Cytidine cytidine | 4 times | Increase | 1.98 |
| monophosphate | | | |
| Dopa | 4 times | Decrease | 0.95 |
| Malic acid | 4 times | Increase | 2.37 |
| S-adenosylhomocysteine | 4 times | Increase | 2.09 |
| S-adenosylmethionine | 4 times | Increase | 1.33 |
| Succinic acid | 4 times | Increase | 1.46 |
| Xanthine | 4 times | Increase | 1.77 |
| Carnitine | 6 times | Increase | 1.30 |
| Citicoline | 6 times | Different from individual | 1.32/0.62 |
| | | to individual | |
| Cytidine | 6 times | Different from individual | 1.54/0.30 |
| | | to individual | |
| Guanosine | 6 times | Decrease | 0.58 |
| Guanosine monophosphate | 6 times | Increase | 1.95 |
| Hypoxanthine | 6 times | Increase | 2.04 |
| Inosine | 6 times | Different from individual | 1.67/0.53 |
| | | to individual | |
| Kynurenine | 6 times | Increase | 1.37 |
| Nicotinamide | 6 times | Increase | 1.32 |
| Serotonin | 6 times | Different from individual | 1.57/0.68 |
| | | to individual | |
| Uridine | 6 times | Increase | 1.31 |
| 4-Hydroxyproline | 10 times | Increase | 1.34 |
| Alanine | 10 times | Increase | 1.34 |
| Asparagine | 10 times | Increase | 1.38 |
| Aspartic acid | 10 times | Increase | 1.76 |
| Cholic acid | 10 times | Increase | 3.26 |
| Citrulline | 10 times | Increase | 1.33 |
| Cystine | 10 times | Decrease | 0.54 |
| Dimethylglycine | 10 times | Increase | 1.37 |
| Glutamic acid | 10 times | Increase | 5.21 |
| Glutamine | 10 times | Increase | 1.34 |
| Glycine | 10 times | Increase | 1.32 |
| Histidine | 10 times | Increase | 1.36 |
| Homocysteine | 10 times | Increase | 1.37 |
| Isoleucine | 10 times | Increase | 1.44 |
| Leucine | 10 times | Increase | 1.35 |
| Pantothenic acid | 10 times | Increase | 1.43 |
| Symmetric | 10 times | Increase | 1.31 |
| dimethylarginine | | | |

Table G presents compounds of which the detection intensity increased or decreased by 30% or more in cases where the time from blood collection to centrifugation, when a serum sample was analyzed by GC/MS, was set to 1 hour, 4 hours, 8 hours, and 12 hours as compared to that in a case where the time was set to 15 minutes. The rate of increase or decrease in Table G is the value of detection intensity relative to 1 that is the detection intensity in a case where the time from blood collection to centrifugation is set to 15 minutes.

TABLE G
compounds affected by time from blood collection
to centrifugation in GC/MS of serum sample

| | Time when compound | | Rate of increase |
|---|---|---|---|
| Compound name | is affected | Increase or decrease | or decrease |
| 2-Aminooctanoic acid | After 4 hours | Increase | 1.60 |
| 2-Hydroxypyridine | After 4 hours | Increase | 2.55 |
| 3-Hydroxyanthranyl acid | After 4 hours | Decrease | 0.59 |
| 3-Hydroxypyruvic acid | After 4 hours | Decrease | 0.58 |
| 3-Indolepropionic acid | After 4 hours | Increase | 1.93 |
| 3-Sulfinoalanine | After 4 hours | Increase | 1.91 |
| Acetylglycine | After 4 hours | Decrease | 0.68 |
| Aconitic acid | After 4 hours | Increase | 1.52 |
| Adenosine | After 4 hours | Decrease | 0.17 |
| Adipic acid | After 4 hours | Different from individual | 2.06/0.61 |
| | | to individual | |
| Allantoin | After 4 hours | Increase | 1.67 |
| Ascorbic acid | After 4 hours | Decrease | 0.62 |
| Azelaic acid | After 4 hours | Decrease | 0.51 |
| Benzoic acid | After 4 hours | Decrease | 1.30 |
| Cadaverine | After 4 hours | Increase | 1.38 |
| Citramalic acid | After 4 hours | Increase | 2.01 |
| Dihydrouracil | After 4 hours | Increase | 3.17 |
| Dihydroxyacetone | After 4 hours | Increase | 1.36 |
| phosphate | | | |
| Dopamine | After 4 hours | Increase | 1.50 |
| Erythrulose | After 4 hours | Decrease | 0.58 |
| Erythrulose | After 4 hours | Decrease | 0.55 |
| Glycerol-3-phosphate | After 4 hours | Increase | 1.39 |
| Glycolic acid | After 4 hours | Increase | 1.40 |
| Hypotaurine | After 4 hours | Increase | 1.69 |
| Hypoxanthine | After 4 hours | Increase | 2.11 |
| Lactitol | After 4 hours | Increase | 3.20 |
| Lactose | After 4 hours | Different from individual | 1.31/0.62 |
| | | to individual | |
| Maleic acid | After 4 hours | Increase | 1.65 |
| Monostearin | After 4 hours | Increase | 1.98 |
| N6-acetyllysine | After 4 hours | Increase | 1.55 |
| Octadecanol | After 4 hours | Increase | 1.32 |
| Oxalic acid | After 4 hours | Increase | 1.31 |
| Pantothenic acid | After 4 hours | Different from individual | 1.78/0.51 |
| | | to individual | |
| Paraxanthine | After 4 hours | Increase | 2.98 |
| Pyridoxamine | After 4 hours | Increase | 1.52 |
| Pyruvic acid | After 4 hours | Decrease | 0.65 |
| Ribose | After 4 hours | Decrease | 0.41 |
| Sorbose | After 4 hours | Increase | 1.53 |
| Sucrose | After 4 hours | Increase | 18.00 |
| Tyramine | After 4 hours | Increase | 2.37 |
| Uracil | After 4 hours | Increase | 1.38 |
| Xylose | After 4 hours | Increase | 1.47 |
| 1,6-Anhydroglucose | After 8 hours | Decrease | 0.62 |

TABLE G
compounds affected by time from blood collection
to centrifugation in GC/MS of serum sample

| | Time when compound | | Rate of increase |
|---|---|---|---|
| Compound name | is affected | Increase or decrease | or decrease |
| 2-Hydroxyglutaric acid | After 8 hours | Increase | 2.00 |
| 2-Ketoisovaleric acid | After 8 hours | Decrease | 0.67 |
| 3-Aminopropionic acid | After 8 hours | Increase | 1.46 |
| (β-alanine) | | | |
| Acetoacetic acid | After 8 hours | Increase | 1.46 |
| Decanoic acid | After 8 hours | Decrease | 0.69 |
| Galacturonic acid | After 8 hours | Decrease | 0.60 |
| Galacturonic acid | After 8 hours | Decrease | 0.64 |
| Glutaric acid | After 8 hours | Increase | 1.39 |
| Inositol | After 8 hours | Increase | 1.46 |
| Lactose | After 8 hours | Different from individual | 3.99/0.65 |
| | | to individual | |
| Meso-erythritol | After 8 hours | Decrease | 0.66 |
| Myo-inositol | After 8 hours | Increase | 1.46 |
| Myristic acid | After 8 hours | Decrease | 1.30 |
| Psicose | After 8 hours | Decrease | 0.65 |
| Putrescine | After 8 hours | Increase | 1.44 |
| Ribitol | After 8 hours | Increase | 1.44 |
| Ribonic acid lactone | After 8 hours | Decrease | 0.68 |
| Ribulose | After 8 hours | Increase | 1.72 |
| Scyllo-inositol | After 8 hours | Decrease | 0.66 |
| Sorbitol | After 8 hours | Increase | 1.54 |
| Threonic acid | After 8 hours | Decrease | 0.55 |
| Trehalose | After 8 hours | Increase | 1.50 |
| Uric acid | After 8 hours | Decrease | 0.69 |
| Xylitol | After 8 hours | Decrease | 0.62 |
| Xylose | After 8 hours | Decrease | 0.68 |
| Xylulose | After 8 hours | Increase | 1.95 |
| 1-Hexadecanol (cetanol) | After 12 hours | Increase | 1.38 |
| 3-Hydroxyisovaleric acid | After 12 hours | Increase | 1.35 |
| 4-Hydroxyproline | After 12 hours | Increase | 1.44 |
| Dihydrouracil | After 12 hours | Decrease | 1.30 |
| Gluconic acid | After 12 hours | Increase | 1.32 |
| N-acetylserine | After 12 hours | Increase | 1.44 |
| Phenylbutyric acid | After 12 hours | Increase | 2.17 |
| Ribonic acid | After 12 hours | Increase | 1.46 |

Table H presents compounds of which the detection intensity increased or decreased by 30% or more in a case where the time from centrifugation to isolation, when a serum sample was analyzed by GC/MS, was set to 1 hour or 6 hours as compared to that in a case where the time was set to 30 minutes. The rate of increase or decrease in Table H is the value of detection intensity relative to 1 that is the detection intensity in a case where the time from centrifugation to isolation is set to 30 minutes.

TABLE H
compounds affected by time from centrifugation to isolation in GC/MS of serum sample

| | Time when compound | | Rate of increase |
|---|---|---|---|
| Compound name | is affected | Increase or decrease | or decrease |
| 1,6-Anhydroglucose | After 1 hour | Increase | 2.25 |
| 1-Hexadecanol (cetanol) | After 1 hour | Increase | 1.36 |
| 2-Aminooctanoic acid | After 1 hour | Increase | 1.67 |
| 2-Hydroxyglutaric acid | After 1 hour | Increase | 1.31 |
| 2-Hydroxypyridine | After 1 hour | Increase | 1.44 |
| 3-Sulfinoalanine | After 1 hour | Increase | 3.07 |
| 4-Hydroxyphenyllactic acid | After 1 hour | Decrease | 0.64 |
| 4-Hydroxyproline | After 1 hour | Increase | 1.33 |
| 5-Hydroxymethyl-2- | After 1 hour | Increase | 1.71 |
| furancarboxylic acid | | | |
| Aconitic acid | After 1 hour | Decrease | 0.04 |
| Adenosine | After 1 hour | Increase | 3.11 |
| Adipic acid | After 1 hour | Increase | 2.50 |
| Azelaic acid | After 1 hour | Increase | 1.71 |
| Benzoic acid | After 1 hour | Decrease | 0.69 |
| Boric acid | After 1 hour | Increase | 2.06 |
| Cadaverine | After 1 hour | Decrease | 0.50 |
| Citramalic acid | After 1 hour | Decrease | 0.33 |
| Dihydrouracil | After 1 hour | Decrease | 0.30 |
| Dopamine | After 1 hour | Decrease | 0.64 |
| Erythrulose | After 1 hour | Increase | 1.31 |
| Galacturonic acid | After 1 hour | Increase | 1.87 |
| Hypoxanthine | After 1 hour | Increase | 1.36 |
| Lactitol | After 1 hour | Increase | 1.60 |
| Lactose | After 1 hour | Increase | 2.37 |
| Maleic acid | After 1 hour | Increase | 1.43 |
| N-acetylserine | After 1 hour | Increase | 1.69 |
| Octadecanol | After 1 hour | Increase | 2.08 |
| Pantothenic acid | After 1 hour | Decrease | 0.66 |
| Phenylbutyric acid | After 1 hour | Increase | 1.75 |
| Psicose | After 1 hour | Decrease | 0.57 |
| Putrescine | After 1 hour | Increase | 1.54 |
| Pyruvic acid | After 1 hour | Decrease | 0.66 |
| Ribitol | After 1 hour | Increase | 2.32 |
| Ribonic acid lactone | After 1 hour | Different from individual | 1.71/0.57 |
| | | to individual | |
| Ribose | After 1 hour | Increase | 1.76 |
| Sucrose | After 1 hour | Increase | 2.12 |
| Trehalose | After 1 hour | Increase | 1.45 |
| 2-Aminobutyric acid | After 6 hours | Increase | 1.60 |
| 3-Hydroxypropionic acid | After 6 hours | Different from individual | 1.91/0.65 |
| | | to individual | |
| 3-Hydroxypyruvic acid | After 6 hours | Increase | 1.88 |
| 3-Indolepropionic acid | After 6 hours | Increase | 1.54 |

TABLE H
compounds affected by time from centrifugation
to isolation in GC/MS of serum sample

| | Time when compound | | Rate of increase |
|---|---|---|---|
| Compound name | is affected | Increase or decrease | or decrease |
| Acetoacetic acid | After 6 hours | Increase | 1.39 |
| Allantoin | After 6 hours | Increase | 3.00 |
| Dihydroxyacetone | After 6 hours | Increase | 1.34 |
| phosphate | | | |
| Glucosamine | After 6 hours | Increase | 1.69 |
| Hydroxylamine | After 6 hours | Increase | 1.87 |
| Lactose | After 6 hours | Increase | 1.43 |
| Monostearin | After 6 hours | Increase | 2.08 |
| N6-acetyllysine | After 6 hours | Different from individual | 1.71/0.58 |
| | | to individual | |
| N-acetylglutamine | After 6 hours | Increase | 1.58 |
| Oxalic acid | After 6 hours | Increase | 1.67 |
| Paraxanthine | After 6 hours | Different from individual | 2.04/0.61 |
| | | to individual | |
| Phenylpyruvic acid | After 6 hours | Increase | 1.45 |
| Pyruvic oxime | After 6 hours | Increase | 1.34 |
| Threonic acid | After 6 hours | Increase | 1.45 |
| Tyramine | After 6 hours | Increase | 3.34 |
| Uracil | After 6 hours | Increase | 4.84 |
| Xylulose | After 6 hours | Increase | 2.48 |

Table I presents compounds of which the detection intensity increased or decreased by 30% or more in cases where the number of times of freezing and thawing, when a serum sample was analyzed by GC/MS, was set to 4 times, 6 times, and 10 times as compared to that in a case where the number of times was set to 2 times. The rate of increase or decrease in Table I is the value of detection intensity relative to 1 that is the detection intensity in a case where the number of times of freezing and thawing is set to 2 times.

TABLE I
compounds affected by number of times of freezing
and thawing in GC/MS of serum sample

| | Number of times when | | Rate of increase |
|---|---|---|---|
| Compound name | compound is affected | Increase or decrease | or decrease |
| 1,6-Anhydroglucose | 4 times | Increase | 2.25 |
| 2-Aminooctanoic acid | 4 times | Increase | 2.80 |
| 2-Hydroxypyridine | 4 times | Decrease | 0.50 |
| 3-Hydroxypropionic acid | 4 times | Decrease | 0.55 |
| 3-Phenyllactic acid | 4 times | Decrease | 0.68 |
| 3-Sulfinoalanine | 4 times | Decrease | 0.47 |
| 4-Hydroxyproline | 4 times | Increase | 1.40 |
| Acetoacetic acid | 4 times | Increase | 1.39 |
| Adenosine | 4 times | Decrease | 0.36 |
| Boric acid | 4 times | Decrease | 0.63 |
| Dihydrouracil | 4 times | Increase | 2.27 |
| Dihydrouracil | 4 times | Decrease | 0.61 |
| Dihydroxyacetone | 4 times | Decrease | 0.65 |
| phosphate | | | |
| Dopamine | 4 times | Decrease | 0.53 |
| Erythrulose | 4 times | Decrease | 0.45 |
| Erythrulose | 4 times | Decrease | 0.46 |
| Glyoxylic acid | 4 times | Decrease | 0.63 |
| Lactose | 4 times | Increase | 1.60 |
| Maleic acid | 4 times | Increase | 2.62 |
| N6-acetyllysine | 4 times | Decrease | 0.67 |
| Oleamide | 4 times | Increase | 2.08 |
| Oxalic acid | 4 times | Decrease | 0.61 |
| Pantothenic acid | 4 times | Increase | 1.39 |
| Phenylbutyric acid | 4 times | Increase | 2.61 |
| Psicose | 4 times | Decrease | 0.54 |
| Ribonic acid lactone | 4 times | Increase | 1.33 |
| Ribose | 4 times | Increase | 1.68 |
| Threonic acid | 4 times | Decrease | 0.56 |
| 3-Hydroxyanthranic acid | 6 times | Increase | 1.34 |
| Allose | 6 times | Decrease | 0.62 |
| Cadaverine | 6 times | Increase | 2.31 |
| Lactose | 6 times | Increase | 1.33 |
| Octadecanol | 6 times | Increase | 1.64 |
| Psicose | 6 times | Increase | 1.30 |
| Uracil | 6 times | Increase | 1.54 |
| 1-Hexadecanol (cetanol) | 10 times | Different from individual | 1.34/0.70 |
| | | to individual | |
| 2-Aminobutyric acid | 10 times | Increase | 2.63 |
| 3-Aminoisobutyric acid | 10 times | Increase | 1.47 |
| 3-Hydroxypyruvic acid | 10 times | Increase | 4.59 |
| 3-Indolepropionic acid | 10 times | Different from individual | 2.21/0.67 |
| | | to individual | |
| Adipic acid | 10 times | Increase | 2.14 |
| Allantoin | 10 times | Increase | 2.94 |
| Arachidonic acid | 10 times | Increase | 1.52 |
| Arginine | 10 times | Increase | 1.51 |

TABLE I
compounds affected by number of times of freezing
and thawing in GC/MS of serum sample

| | Number of times when | | Rate of increase |
|---|---|---|---|
| Compound name | compound is affected | Increase or decrease | or decrease |
| Azelaic acid | 10 times | Increase | 9.39 |
| Benzoic acid | 10 times | Increase | 3.86 |
| Cabroic acid | 10 times | Increase | 1.32 |
| Citramalic acid | 10 times | Decrease | 0.53 |
| Docosahexaenoic acid | 10 times | Increase | 1.58 |
| Eicosapentaenoic acid | 10 times | Increase | 1.51 |
| Glucosamine | 10 times | Increase | 2.43 |
| Glycolic acid | 10 times | Decrease | 0.66 |
| Hydroxylamine | 10 times | Increase | 2.31 |
| Hypoxanthine | 10 times | Different from individual | 1.47/0.66 |
| | | to individual | |
| Margaric acid | 10 times | Increase | 1.35 |
| Meso-erythritol | 10 times | Decrease | 0.69 |
| Monostearin | 10 times | Decrease | 0.60 |
| N-acetylglutamine | 10 times | Increase | 2.17 |
| Pelargonic acid | 10 times | Decrease | 0.67 |
| Paraxanthine | 10 times | Different from individual | 2.65/0.63 |
| | | to individual | |
| Phenylpyruvic acid | 10 times | Increase | 2.06 |
| Putrescine | 10 times | Increase | 1.77 |
| Pyridoxamine | 10 times | Decrease | 0.61 |
| Pyruvic oxime | 10 times | Increase | 1.69 |
| Ribulose | 10 times | Decrease | 0.68 |
| Sarcosine | 10 times | Increase | 1.32 |
| Sorbitol | 10 times | Decrease | 0.61 |
| Sorbose | 10 times | Decrease | 0.68 |
| Stearic acid | 10 times | Increase | 1.33 |
| Sucrose | 10 times | Decrease | 0.64 |
| Trehalose | 10 times | Decrease | 0.69 |
| Tyramine | 10 times | Increase | 3.78 |
| Uric acid | 10 times | Different from individual | 1.33/0.66 |
| | | to individual | |

Table J presents compounds of which the detection intensity increased or decreased by 30% or more in cases where the time from blood collection to centrifugation, when a serum sample was analyzed by LC/MS, was set to 1 hour, 4 hours, 8 hours, and 12 hours as compared to that in a case where the time was set to 15 minutes. The rate of increase or decrease in Table J is the value of detection intensity relative to 1 that is the detection intensity in a case where the time from blood collection to centrifugation is set to 15 minutes.

TABLE J
compounds affected by time from blood collection
to centrifugation in LC/MS of serum sample

| | Time when compound | | Rate of increase |
|---|---|---|---|
| Compound name | is affected | Increase or decrease | or decrease |
| Adenosine | After 4 hours | Decrease | 0.29 |
| Adenosine 3′,5′-cyclic | After 4 hours | Decrease | 0.60 |
| monophosphate | | | |
| Allantoin | After 4 hours | Different from individual | 1.78/0.40 |
| | | to individual | |
| Aspartic acid | After 4 hours | Increase | 2.10 |
| Carnosine | After 4 hours | Increase | 2.83 |
| Choline | After 4 hours | Increase | 1.48 |
| Cytidine | After 4 hours | Decrease | 0.62 |
| Dopa | After 4 hours | Decrease | 0.56 |
| Glutamic acid | After 4 hours | Increase | 3.09 |
| Guanosine | After 4 hours | Decrease | 0.08 |
| Guanosine 3′,5′-cyclic | After 4 hours | Different from individual | 3.62/0.58 |
| monophosphate | | to individual | |
| Hypoxanthine | After 4 hours | Increase | 2.24 |
| Inosine | After 4 hours | Decrease | 0.17 |
| Malic acid | After 4 hours | Increase | 1.33 |
| Nicotinamide | After 4 hours | Increase | 2.16 |
| Ornithine | After 4 hours | Increase | 1.48 |
| S-adenosylhomocysteine | After 4 hours | Increase | 2.82 |
| Uridine | After 4 hours | Increase | 1.42 |
| Xanthine | After 4 hours | Increase | 1.92 |
| Arginine | After 6 hours | Decrease | 0.54 |
| Argininosuccinic acid | After 6 hours | Decrease | 0.65 |
| Cysteine | After 6 hours | Increase | 1.39 |
| Methionine sulfoxide | After 6 hours | Increase | 1.65 |
| Serine | After 6 hours | Increase | 1.44 |
| Succinic acid | After 6 hours | Increase | 1.57 |
| Asparagine | After 12 hours | Increase | 1.40 |
| Proline | After 12 hours | Increase | 1.32 |
| Histidine | After 12 hours | Increase | 1.46 |
| Pantothenic acid | After 12 hours | Increase | 1.43 |
| Isoleucine | After 12 hours | Increase | 1.34 |
| Leucine | After 12 hours | Increase | 1.35 |
| Dopamine | After 12 hours | Increase | 1.39 |
| Glycine | After 12 hours | Increase | 1.50 |

Table K presents compounds of which the detection intensity increased or decreased by 30% or more in a case where the time from centrifugation to isolation, when a serum sample was analyzed by LC/MS, was set to 1 hour or 6 hours as compared to that in a case where the time was set to 30 minutes. The rate of increase or decrease in Table K is the value of detection intensity relative to 1 that is the detection intensity in a case where the time from centrifugation to isolation is set to 30 minutes.

TABLE K
compounds affected by time from centrifugation to isolation in LC/MS of serum sample

| | Time when compound | | Rate of increase |
|---|---|---|---|
| Compound name | is affected | Increase or decrease | or decrease |
| Adenine | After 1 hour | Decrease | 0.39 |
| Adenosine | After 1 hour | Decrease | 0.07 |
| Adenosine monophosphate | After 1 hour | Decrease | 0.13 |
| Argininosuccinic acid | After 1 hour | Increase | 1.32 |
| Carnosine | After 1 hour | Different from individual | 1.41/0.56 |
| | | to individual | |
| Cystine | After 1 hour | Decrease | 0.67 |
| Cytidine | After 1 hour | Decrease | 0.70 |
| Glutamic acid | After 1 hour | Decrease | 0.53 |
| Guanosine | After 1 hour | Decrease | 0.57 |
| Guanosine 3′,5′-cyclic | After 1 hour | Different from individual | 1.49/0.46 |
| monophosphate | | to individual | |
| Inosine | After 1 hour | Decrease | 0.61 |
| Malic acid | After 1 hour | Increase | 1.38 |
| S-adenosylhomocysteine | After 1 hour | Increase | 1.51 |
| Serotonin | After 1 hour | Decrease | 0.52 |
| Adenosine 3′,5′-cyclic | After 6 hours | Decrease | 0.60 |
| monophosphate | | | |
| Allantoin | After 6 hours | Increase | 10.46 |
| Aspartic acid | After 6 hours | Increase | 2.40 |
| Cysteine | After 6 hours | Decrease | 0.61 |
| Hypoxanthine | After 6 hours | Increase | 2.78 |
| Methionine sulfoxide | After 6 hours | Increase | 1.77 |
| Proline | After 6 hours | Increase | 2.35 |
| Xanthine | After 6 hours | Increase | 2.50 |

Table M presents compounds of which the detection intensity increased or decreased by 30% or more in cases where the number of times of freezing and thawing, when a serum sample was analyzed by LC/MS, was set to 4 times, 6 times, and 10 times as compared to that in a case where the number of times was set to 2 times. The rate of increase or decrease in Table M is the value of detection intensity relative to 1 that is the detection intensity in a case where the number of times of freezing and thawing is set to 2 times.

TABLE M
compounds affected by number of times of freezing
and thawing in LC/MS of serum sample

| | Number of times when | | Rate of increase |
|---|---|---|---|
| Compound name | compound is affected | Increase or decrease | or decrease |
| Adenine | 4 times | Decrease | 0.57 |
| Adenosine | 4 times | Decrease | 0.54 |
| Adenosine 3′,5′-cyclic | 4 times | Increase | 1.32 |
| monophosphate | | | |
| Adenosine monophosphate | 4 times | Decrease | 0.23 |
| Allantoin | 4 times | Increase | 3.27 |
| Carnosine | 4 times | Decrease | 0.59 |
| Creatine | 4 times | Decrease | 0.63 |
| Cysteine | 4 times | Increase | 1.37 |
| Cystine | 4 times | Increase | 1.56 |
| Cytidine | 4 times | Increase | 1.52 |
| Guanosine 3′,5′-cyclic | 4 times | Increase | 1.47 |
| monophosphate | | | |
| Hypoxanthine | 4 times | Different from individual | 1.35/0.61 |
| | | to individual | |
| Inosine | 4 times | Increase | 1.44 |
| Kynurenine | 4 times | Decrease | 0.67 |
| Methionine sulfoxide | 4 times | Increase | 1.55 |
| Succinic acid | 4 times | Increase | 1.47 |
| Uridine | 4 times | Decrease | 0.68 |
| Xanthine | 4 times | Different from individual | 1.37/0.70 |
| | | to individual | |
| 2-Aminobutyric acid | 6 times | Different from individual | 1.31/0.61 |
| | | to individual | |
| 4-Hydroxyproline | 6 times | Decrease | 0.66 |
| Alanine | 6 times | Different from individual | 1.32/0.63 |
| | | to individual | |
| Arginine | 6 times | Decrease | 0.64 |
| Argininosuccinic acid | 6 times | Different from individual | 1.38/0.49 |
| | | to individual | |
| Asparagine | 6 times | Decrease | 0.66 |
| Asymmetric | 6 times | Decrease | 0.69 |
| dimethylarginine | | | |
| Carnitine | 6 times | Decrease | 0.67 |
| Cholic acid | 6 times | Decrease | 0.57 |
| Choline | 6 times | Decrease | 0.68 |
| Citrulline | 6 times | Decrease | 0.62 |
| Creatinine | 6 times | Decrease | 0.67 |
| Dimethylglycine | 6 times | Different from individual | 1.34/0.61 |
| | | to individual | |
| Dopa | 6 times | Different from individual | 1.30/0.31 |
| | | to individual | |
| Glycine | 6 times | Decrease | 0.69 |
| Guanosine | 6 times | Decrease | 0.62 |
| Histidine | 6 times | Decrease | 0.61 |
| Homocysteine | 6 times | Decrease | 1.30 |
| Isoleucine | 6 times | Different from individual | 1.33/0.66 |
| | | to individual | |
| Leucine | 6 times | Decrease | 0.66 |
| Methionine | 6 times | Decrease | 0.69 |
| Nicotinamide | 6 times | Decrease | 0.69 |
| S-adenosylhomocysteine | 6 times | Different from individual | 1.62/0.49 |
| | | to individual | |
| Serine | 6 times | Decrease | 0.66 |
| Symmetric | 6 times | Decrease | 0.55 |
| dimethylarginine | | | |
| Threonine | 6 times | Different from individual | 1.31/0.69 |
| | | to individual | |
| Tryptophan | 6 times | Decrease | 0.66 |
| Tyrosine | 6 times | Decrease | 0.60 |
| Uric acid | 6 times | Decrease | 0.70 |
| Acetylcarnitine | 10 times | Decrease | 0.70 |
| Aspartic acid | 10 times | Decrease | 0.53 |
| Glutamic acid | 10 times | Decrease | 0.63 |
| Malic acid | 10 times | Decrease | 0.64 |
| Ornithine | 10 times | Increase | 1.31 |
| Pantothenic acid | 10 times | Decrease | 0.55 |
| Phenylalanine | 10 times | Decrease | 0.57 |
| Proline | 10 times | Decrease | 0.59 |
| Serotonin | 10 times | Decrease | 0.65 |
| Valine | 10 times | Decrease | 0.56 |

The disclosed contents of the next application as the basis of priority are incorporated herein by reference.

Japanese Patent Application No. 2019-089363 (filed on May 9, 2019)

1. A method for evaluating a sample, which comprises:
obtaining a blood plasma sample prepared from human blood;
conducting detection of at least one molecule selected from the group consisting of 1,6-anhydroglucose, 1-hexadecanol, 2-aminobutyric acid, 2-ketobutyric acid, 2′-deoxyuridine, 2-hydroxyisocaproic acid, 2-hydroxypyridine, 3-aminoisobutyric acid, 3-sulfinoalanine, 3-phenyllactic acid, 4-aminobutyric acid, 4-hydroxyphenyllactic acid, 4-hydroxyproline, 5-glutamylcysteine, N6-acetyllysine, N-acetylserine, S-adenosylhomocysteine, S-adenosylmethionine, aconitic acid, ascorbic acid, asparagine, aspartic acid, acetylcarnitine, azelaic acid, adenine, adenosine, adenosine monophosphate, adenosine 3′,5′-cyclic monophosphate, arachidonic acid, alanine, allantoin, argininosuccinic acid, arginine, isoleucine, inosine, indoxyl sulfate, uridine, octadecanol, ornithine, oleic acid, cabroic acid, galacturonic acid, carnitine, xanthine, xylose, kynurenine, quinolinic acid, guanosine, guanosine monophosphate, glyoxylic acid, glycolic acid, glycine, glycerol-3-phosphate, glutamine, glutamic acid, creatinine, creatine, cholic acid, succinic acid, choline, cholesterol, cystathionine, cystine, cysteine, citicoline, cytidine, cytidine monophosphate, cytosine, citrulline, dihydrouracil, dihydroxyacetone phosphate, dimethylglycine, oxalic acid, scyllo-inositol, sucrose, stearic acid, serotonin, sorbose, symmetric dimethylarginine, dopa, dopamine, docosahexaenoic acid, tryptamine, tryptophan, trehalose, nicotinamide, uric acid, paraxanthine, palmitic acid, pantothenic acid, histamine, histidine, asymmetric dimethylarginine, hydroquinone, hypoxanthine, hypoxanthine, hypotaurine, psicose, proline, boric acid, homocysteine, maleic acid, mannose, myristic acid, methionine sulfoxide, methionine sulfone, monostearin, lactitol, lactose, linoleic acid, ribulose, ribose, ribonic acid, malic acid, leucine, and uric acid in the blood plasma sample; and
evaluating quality of the blood plasma sample based on intensity of the molecule acquired by the detection.

2. The method for evaluating a sample according to claim 1, wherein
detection of at least one molecule selected from the group consisting of 1,6-anhydroglucose, 1-hexadecanol, 2-ketobutyric acid, 2′-deoxyuridine, 2-hydroxyisocaproic acid, 2-hydroxypyridine, 3-aminoisobutyric acid, 3-sulfinoalanine, 3-phenyllactic acid, 4-hydroxyphenyllactic acid, N6-acetyllysine, N-acetylserine, aconitic acid, ascorbic acid, azelaic acid, allantoin, indoxyl sulfate, uridine, octadecanol, oleic acid, cabroic acid, galacturonic acid, xanthine, xylose, quinolinic acid, glyoxylic acid, glycolic acid, glycerol-3-phosphate, creatinine, cholesterol, cytosine, dihydrouracil, dihydroxyacetone phosphate, dimethylglycine, oxalic acid, scyllo-inositol, sucrose, stearic acid, sorbose, docosahexaenoic acid, tryptamine, trehalose, uric acid, paraxanthine, palmitic acid, pantothenic acid, histamine, hydroquinone, hypotaurine, psicose, boric acid, maleic acid, mannose, myristic acid, methionine sulfone, monostearin, lactitol, lactose, linoleic acid, ribulose, ribose, ribonic acid, malic acid, and uric acid in the blood plasma sample is conducted by gas chromatography/mass spectrometry in the detection.

3. The method for evaluating a sample according to claim 2, wherein
detection of at least one molecule selected from the group consisting of 1,6-anhydroglucose, 1-hexadecanol, 2-hydroxypyridine, 2-ketobutyric acid, 3-sulfinoalanine, aconitic acid, allantoin, arachidonic acid, ascorbic acid, azelaic acid, cytosine, dihydroxyacetone phosphate, glycerol-3-phosphate, histamine, hydroquinone, lactitol, maleic acid, mannose, methionine sulfone, N-acetylserine, octadecanol, oxalic acid, pantothenic acid, psicose, quinolinic acid, ribonic acid, ribulose, sorbose, sucrose, uridine, xanthine, xylose, docosahexaenoic acid, hypotaurine, trehalose, 2′-deoxyuridine, 3-aminoisobutyric acid, 4-hydroxyphenyllactic acid, cholesterol, dimethylglycine, indoxyl sulfate, lactose, linoleic acid, malic acid, monostearin, myristic acid, oleic acid, palmitic acid, stearic acid, and uric acid in the blood plasma sample is conducted in the detection, and
quality of the blood plasma sample based on time from when the blood is collected until the blood is subjected to centrifugation is evaluated based on intensity of the molecule acquired by the detection.

4. The method for evaluating a sample according to claim 2, wherein
detection of at least one molecule selected from the group consisting of 1,6-anhydroglucose, 2′-deoxyuridine, 2-hydroxyisocaproic acid, 2-hydroxypyridine, 2-ketobutyric acid, 3-sulfinoalanine, 3-phenyllactic acid, allantoin, azelaic acid, dihydrouracil, dihydroxyacetone phosphate, docosahexaenoic acid, glycerol-3-phosphate, glycolic acid, glyoxylic acid, histamine, hydroquinone, hypotaurine, lactitol, lactose, maleic acid, mannose, methionine sulfone, N6-acetyllysine, N-acetylserine, oxalic acid, pantothenic acid, paraxanthine, psicose, quinolinic acid, ribose, ribulose, sucrose, trehalose, uric acid, uridine, and xanthine in the blood plasma sample is conducted in the detection, and
quality of the blood plasma sample based on time from when the blood is collected until the blood is subjected to cooling is evaluated based on intensity of the molecule acquired by the detection.

5. The method for evaluating a sample according to claim 2, wherein
detection of at least one molecule selected from the group consisting of 1,6-anhydroglucose, 2-hydroxypyridine, 3-sulfinoalanine, ascorbic acid, azelaic acid, boric acid, cabroic acid, galacturonic acid, hydroquinone, lactose, methionine sulfone, pantothenic acid, psicose, quinolinic acid, ribonic acid, ribulose, sucrose, 2′-deoxyuridine, 2-hydroxyisocaproic acid, cytosine, dihydroxyacetone phosphate, glycerol-3-phosphate, indoxyl sulfate, mannose, monostearin, N6-acetyllysine, N-acetylserine, octadecanol, ribose, scyllo-inositol, trehalose, uridine, xanthine, xylose, 1-hexadecanol, cetanol, 3-phenyllactic acid, allantoin, creatinine, dimethylglycine, histamine, lactitol, maleic acid, and tryptamine in the blood plasma sample is conducted in the detection, and
quality of the blood plasma sample based on a number of times by which the blood plasma sample is subjected to freezing and thawing is evaluated based on intensity of the molecule acquired by the detection.

6. The method for evaluating a sample according to claim 1, wherein
detection of at least one molecule selected from the group consisting of 2-aminobutyric acid, 4-aminobutyric acid, 4-hydroxyproline, 5-glutamylcysteine, S-adenosylhomocysteine, S-adenosylmethionine, asparagine, aspartic acid, acetylcarnitine, adenine, adenosine, adenosine monophosphate, adenosine 3′,5′-cyclic monophosphate, alanine, allantoin, argininosuccinic acid, arginine, isoleucine, inosine, uridine, ornithine, carnitine, xanthine, kynurenine, guanosine, guanosine monophosphate, glycine, glutamine, glutamic acid, creatinine, creatine, cholic acid, succinic acid, choline, cystathionine, cystine, cysteine, citicoline, cytidine, cytidine monophosphate, citrulline, dimethylglycine, serotonin, symmetric dimethylarginine, symmetric dimethylarginine, dopa, dopamine, tryptophan, nicotinamide, pantothenic acid, histidine, asymmetric dimethylarginine, hypoxanthine, proline, homocysteine, methionine sulfoxide, malic acid, leucine, and uric acid in the blood plasma sample by liquid chromatography/mass spectrometry is conducted in the detection.

7. The method for evaluating a sample according to claim 6, wherein
detection of at least one molecule selected from the group consisting of 5-glutamylcysteine, adenosine, adenosine monophosphate, allantoin, citicoline, cysteine, cytidine, cytidine monophosphate, dopa, guanosine monophosphate, hypoxanthine, inosine, nicotinamide, proline, S-adenosylhomocysteine, serotonin, succinic acid, 4-aminobutyric acid, adenine, arginine, aspartic acid, dopamine, guanosine, malic acid, pantothenic acid, S-adenosylmethionine, succinic acid, xanthine, 2-aminobutyric acid, 4-hydroxyproline, acetylcarnitine, adenosine 3′,5′-cyclic monophosphate, alanine, argininosuccinic acid, asymmetric dimethylarginine, carnitine, cholic acid, choline, citrulline, creatine, creatinine, cystathionine, cystine, dimethylglycine, isoleucine, kynurenine, leucine, methionine sulfoxide, symmetric dimethylarginine, tryptophan, uric acid, and uridine in the blood plasma sample is conducted in the detection, and
quality of the blood plasma sample based on time from when the blood is collected until the blood is subjected to centrifugation is evaluated based on intensity of the molecule acquired by the detection.

8. The method for evaluating a sample according to claim 6, wherein
detection of at least one molecule selected from the group consisting of 4-aminobutyric acid, 5-glutamylcysteine, adenine, adenosine, adenosine monophosphate, allantoin, aspartic acid, asymmetric dimethylarginine, cholic acid, choline, citicoline, cysteine, cytidine, cytidine monophosphate, dimethylglycine, dopa, dopamine, guanosine monophosphate, hypoxanthine, inosine, nicotinamide, ornithine, proline, S-adenosylhomocysteine, S-adenosylmethionine, serotonin, and xanthine in the blood plasma sample is conducted in the detection, and
quality of the blood plasma sample based on time from when the blood is collected until the blood is subjected to cooling is evaluated based on intensity of the molecule acquired by the detection.

9. The method for evaluating a sample according to claim 6, wherein
detection of at least one molecule selected from the group consisting of 4-aminobutyric acid, 5-glutamylcysteine, adenine, adenosine, adenosine monophosphate, allantoin, arginine, argininosuccinic acid, choline, creatine, creatinine, cystathionine, cysteine, cytidine monophosphate, dopa, malic acid, S-adenosylhomocysteine, S-adenosylmethionine, succinic acid, xanthine, carnitine, citicoline, cytidine, guanosine, guanosine monophosphate, hypoxanthine, inosine, kynurenine, nicotinamide, serotonin, uridine, 4-hydroxyproline, alanine, asparagine, aspartic acid, cholic acid, citrulline, cystine, dimethylglycine, glutamic acid, glutamine, glycine, histidine, homocysteine, isoleucine, leucine, pantothenic acid, and symmetric dimethylarginine in the blood plasma sample is conducted in the detection, and
quality of the blood plasma sample based on a number of times by which the blood plasma sample is subjected to freezing and thawing is evaluated based on intensity of the molecule acquired by the detection.

10. An analysis method, which comprises:
conducting evaluation of a blood plasma sample by the method for evaluating a sample according to claim 1; and
conducting analysis of a blood plasma sample based on the evaluation.

11. A method for detecting a degraded sample, which comprises:
obtaining a blood plasma sample prepared from human blood; and
conducting detection of at least one molecule selected from the group consisting of 1,6-anhydroglucose, 1-hexadecanol, 2-aminobutyric acid, 2-ketobutyric acid, 2′-deoxyuridine, 2-hydroxyisocaproic acid, 2-hydroxypyridine, 3-aminoisobutyric acid, 3-sulfinoalanine, 3-phenyllactic acid, 4-aminobutyric acid, 4-hydroxyphenyllactic acid, 4-hydroxyproline, 5-glutamylcysteine, N6-acetyllysine, N-acetylserine, S-adenosylhomocysteine, S-adenosylmethionine, aconitic acid, ascorbic acid, asparagine, aspartic acid, acetylcarnitine, azelaic acid, adenine, adenosine, adenosine monophosphate, adenosine 3′,5′-cyclic monophosphate, arachidonic acid, alanine, allantoin, argininosuccinic acid, arginine, isoleucine, inosine, indoxyl sulfate, uridine, octadecanol, ornithine, oleic acid, cabroic acid, galacturonic acid, carnitine, xanthine, xylose, kynurenine, quinolinic acid, guanosine, guanosine monophosphate, glyoxylic acid, glycolic acid, glycine, glycerol-3-phosphate, glutamine, glutamic acid, creatinine, creatine, cholic acid, succinic acid, choline, cholesterol, cystathionine, cystine, cysteine, citicoline, cytidine, cytidine monophosphate, cytosine, citrulline, dihydrouracil, dihydroxyacetone phosphate, dimethylglycine, oxalic acid, scyllo-inositol, sucrose, stearic acid, serotonin, sorbose, symmetric dimethylarginine, dopa, dopamine, docosahexaenoic acid, tryptamine, tryptophan, trehalose, nicotinamide, uric acid, paraxanthine, palmitic acid, pantothenic acid, histamine, histidine, asymmetric dimethylarginine, hydroquinone, hypoxanthine, hypoxanthine, hypotaurine, psicose, proline, boric acid, homocysteine, maleic acid, mannose, myristic acid, methionine sulfoxide, methionine sulfone, monostearin, lactitol, lactose, linoleic acid, ribulose, ribose, ribonic acid, malic acid, leucine, and uric acid in the blood plasma sample.

12. A marker for detecting a degraded blood plasma sample, which comprises at least one molecule selected from the group consisting of 1,6-anhydroglucose, 1-hexadecanol, 2-aminobutyric acid, 2-ketobutyric acid, 2′-deoxyuridine, 2-hydroxyisocaproic acid, 2-hydroxypyridine, 3-aminoisobutyric acid, 3-sulfinoalanine, 3-phenyllactic acid, 4-aminobutyric acid, 4-hydroxyphenyllactic acid, 4-hydroxyproline, 5-glutamylcysteine, N6-acetyllysine, N-acetylserine, S-adenosylhomocysteine, S-adenosylmethionine, aconitic acid, ascorbic acid, asparagine, aspartic acid, acetylcarnitine, azelaic acid, adenine, adenosine, adenosine monophosphate, adenosine 3′,5′-cyclic monophosphate, arachidonic acid, alanine, allantoin, argininosuccinic acid, arginine, isoleucine, inosine, indoxyl sulfate, uridine, octadecanol, ornithine, oleic acid, cabroic acid, galacturonic acid, carnitine, xanthine, xylose, kynurenine, quinolinic acid, guanosine, guanosine monophosphate, glyoxylic acid, glycolic acid, glycine, glycerol-3-phosphate, glutamine, glutamic acid, creatinine, creatine, cholic acid, succinic acid, choline, cholesterol, cystathionine, cystine, cysteine, citicoline, cytidine, cytidine monophosphate, cytosine, citrulline, dihydrouracil, dihydroxyacetone phosphate, dimethylglycine, oxalic acid, scyllo-inositol, sucrose, stearic acid, serotonin, sorbose, symmetric dimethylarginine, dopa, dopamine, docosahexaenoic acid, tryptamine, tryptophan, trehalose, nicotinamide, uric acid, paraxanthine, palmitic acid, pantothenic acid, histamine, histidine, asymmetric dimethylarginine, hydroquinone, hypoxanthine, hypoxanthine, hypotaurine, psicose, proline, boric acid, homocysteine, maleic acid, mannose, myristic acid, methionine sulfoxide, methionine sulfone, monostearin, lactitol, lactose, linoleic acid, ribulose, ribose, ribonic acid, malic acid, leucine, and uric acid.

13. A method for evaluating a sample, which comprises:
obtaining a serum sample prepared from human blood;
conducting detection of at least one molecule selected from the group consisting of 1,6-anhydroglucose, 1-hexadecanol, 2-aminooctanoic acid, 2-aminobutyric acid, 2-ketoisovaleric acid, 2-hydroxyglutaric acid, 2-hydroxypyridine, 3-aminoisobutyric acid, 3-aminopropionic acid, β-alanine, 3-indolepropionic acid, 3-sulfinoalanine, 3-hydroxyanthranyl acid, 3-hydroxyisovaleric acid, 3-hydroxypyruvic acid, 3-hydroxypropionic acid, 3-phenyllactic acid, 4-hydroxyphenyllactic acid, 4-hydroxyproline, 5-hydroxymethyl-2-furancarboxylic acid, N6-acetyllysine, N-acetylglutamine, N-acetylserine, S-adenosylhomocysteine, aconitic acid, adipic acid, ascorbic acid, asparagine, aspartic acid, acetylcarnitine, acetylglycine, acetoacetic acid, azelaic acid, adenine, adenosine, adenosine monophosphate, adenosine 3′,5′-cyclic monophosphate, arachidonic acid, alanine, allantoin, argininosuccinic acid, arginine, allose, benzoic acid, isoleucine, inositol, inosine, uracil, uridine, eicosapentaenoic acid, erythrulose, octadecanol, ornithine, oleamide, cadaverine, cabroic acid, galacturonic acid, carnitine, carnosine, xanthine, xylitol, xylulose, xylose, kynurenine, guanosine, guanosine 3′,5′-cyclic monophosphate, glyoxylic acid, glycolic acid, glycine, glycerol-3-phosphate, glucosamine, gluconic acid, glutamic acid, glutaric acid, creatinine, creatine, cholic acid, succinic acid, choline, sarcosine, cystine, cysteine, cytidine, citramalic acid, citrulline, dihydrouracil, dihydroxyacetone phosphate, dimethylglycine, oxalic acid, scyllo-inositol, sucrose, stearic acid, serine, serotonin, sorbitol, sorbose, tyramine, tyrosine, decanoic acid, dopa, dopamine, docosahexaenoic acid, tryptophan, threonine, threonic acid, trehalose, nicotinamide, paraxanthine, valine, pantothenic acid, histidine, asymmetric dimethylarginine, hydroxylamine, hypoxanthine, hypotaurine, pyridoxamine, pyruvic oxime, pyruvic acid, phenylalanine, phenylpyruvic acid, phenylbutyric acid, psicose, putrescine, proline, pelargonic acid, boric acid, homocysteine, margaric acid, maleic acid, myo-inositol, myristic acid, meso-erythritol, methionine, methionine sulfoxide, monostearin, lactitol, lactose, ribitol, ribulose, ribose, ribonic acid, ribonic acid lactone, malic acid, leucine, benzoic acid, symmetric dimethylarginine, and uric acid in the serum sample; and
evaluating quality of the serum sample based on intensity of the molecule acquired by the detection.

14. The method for evaluating a sample according to claim 13, wherein
detection of at least one molecule selected from the group consisting of 1,6-anhydroglucose, 1-hexadecanol, 2-aminooctanoic acid, 2-aminobutyric acid, 2-ketoisovaleric acid, 2-hydroxyglutaric acid, 2-hydroxypyridine, 3-aminoisobutyric acid, 3-aminopropionic acid, 3-indolepropionic acid, 3-sulfinoalanine, 3-hydroxyanthranylic acid, 3-hydroxyisovaleric acid, 3-hydroxypyruvic acid, 3-hydroxypropionic acid, 3-phenyllactic acid, 4-hydroxyphenyllactic acid, 4-hydroxyproline, 5-hydroxymethyl-2-furancarboxylic acid, N6-acetyllysine, N-acetylglutamine, N-acetylserine, aconitic acid, adipic acid, ascorbic acid, acetylglycine, acetoacetic acid, azelaic acid, adenosine, arachidonic acid, allantoin, arginine, allose, benzoic acid, inositol, uracil, eicosapentaenoic acid, erythrulose, octadecanol, oleamide, cadaverine, cabroic acid, galacturonic acid, xylitol, xylulose, xylose, glyoxylic acid, glycolic acid, glycerol-3-phosphate, glucosamine, gluconic acid, glutaric acid, sarcosine, citramalic acid, dihydrouracil, dihydroxyacetone phosphate, oxalic acid, scyllo-inositol, sucrose, stearic acid, sorbitol, sorbose, tyramine, decanoic acid, dopamine, docosahexaenoic acid, threonic acid, trehalose, paraxanthine, pantothenic acid, hydroxylamine, hypoxanthine, hypotaurine, pyridoxamine, pyruvic oxime, pyruvic acid, phenylpyruvic acid, phenylbutyric acid, psicose, putrescine, pelargonic acid, boric acid, margaric acid, maleic acid, myo-inositol, myristic acid, meso-erythritol, monostearin, lactitol, lactose, ribitol, ribulose, ribose, ribonic acid, ribonic acid lactone, benzoic acid, and uric acid in the serum sample by gas chromatography/mass spectrometry is conducted.

15. The method for evaluating a sample according to claim 14, wherein
detection of at least one molecule selected from the group consisting of 2-aminooctanoic acid, 2-hydroxypyridine, 3-hydroxyanthranyl acid, 3-hydroxypyruvic acid, 3-indolepropionic acid, 3-sulfinoalanine, acetylglycine, aconitic acid, adenosine, adipic acid, allantoin, ascorbic acid, azelaic acid, benzoic acid, cadaverine, citramalic acid, dihydrouracil, dihydroxyacetone phosphate, dopamine, erythrulose, glycerol-3-phosphate, glycolic acid, hypotaurine, hypoxanthine, lactitol, lactose, maleic acid, monostearin, N6-acetyllysine, octadecanol, oxalic acid, pantothenic acid, paraxanthine, pyridoxamine, pyruvic acid, ribose, sorbose, sucrose, tyramine, uracil, xylose, 1,6-anhydroglucose, 2-hydroxyglutaric acid, 2-ketoisovaleric acid, 3-aminopropionic acid, acetoacetic acid, decanoic acid, galacturonic acid, galacturonic acid, glutaric acid, inositol, lactose, meso-erythritol, myo-inositol, myristic acid, psicose, putrescine, ribitol, ribonic acid lactone, ribulose, scyllo-inositol, sorbitol, threonic acid, trehalose, uric acid, xylitol, xylose, xylulose, 1-hexadecanol, 3-hydroxyisovaleric acid, 4-hydroxyproline, dihydrouracil, gluconic acid, N-acetylserine, phenylbutyric acid, and ribonic acid in the serum sample is conducted in the detection, and
quality of the serum sample based on time from when the blood is collected until the blood is subjected to centrifugation is evaluated based on intensity of the molecule acquired by the detection.

16. The method for evaluating a sample according to claim 14, wherein
detection of at least one molecule selected from the group consisting of 1,6-anhydroglucose, 1-hexadecanol, 2-aminooctanoic acid, 2-hydroxyglutaric acid, 2-hydroxypyridine, 3-sulfinoalanine, 4-hydroxyphenyllactic acid, 4-hydroxyproline, 5-hydroxymethyl-2-furancarboxylic acid, aconitic acid, adenosine, adipic acid, azelaic acid, benzoic acid, boric acid, cadaverine, citramalic acid, dihydrouracil, dopamine, erythrulose, galacturonic acid, hypoxanthine, lactitol, lactose, maleic acid, N-acetylserine, octadecanol, pantothenic acid, phenylbutyric acid, psicose, putrescine, pyruvic acid, ribitol, ribonic acid lactone, ribose, sucrose, trehalose, 2-aminobutyric acid, 3-hydroxypropionic acid, 3-hydroxypyruvic acid, 3-indolepropionic acid, acetoacetic acid, allantoin, dihydroxyacetone phosphate, glucosamine, hydroxylamine, lactose, monostearin, N6-acetyllysine, N-acetylglutamine, oxalic acid, paraxanthine, phenylpyruvic acid, pyruvic oxime, threonic acid, tyramine, uracil, and xylulose in the serum sample is conducted in the detection, and
quality of the serum sample based on time from when centrifugation of the blood is conducted until serum obtained by the centrifugation is isolated is evaluated based on intensity of the molecule acquired by the detection.

17. The method for evaluating a sample according to claim 14, wherein
detection of at least one molecule selected from the group consisting of 1,6-anhydroglucose, 2-aminooctanoic acid, 2-hydroxypyridine, 3-hydroxypropionic acid, 3-phenyllactic acid, 3-sulfinoalanine, 4-hydroxyproline, acetoacetic acid, adenosine, boric acid, dihydrouracil, dihydrouracil, dihydroxyacetone phosphate, dopamine, erythrulose, erythrulose, glyoxylic acid, lactose, maleic acid, N6-acetyllysine, oleamide, oxalic acid, pantothenic acid, phenylbutyric acid, psicose, ribonic acid lactone, ribose, threonic acid, 3-hydroxyanthranic acid, allose, cadaverine, lactose, octadecanol, psicose, uracil, 1-hexadecanol, 2-aminobutyric acid, 3-aminoisobutyric acid, 3-hydroxypyruvic acid, 3-indolepropionic acid, adipic acid, allantoin, arachidonic acid, arginine, azelaic acid, benzoic acid, cabroic acid, citramalic acid, docosahexaenoic acid, eicosapentaenoic acid, glucosamine, glycolic acid, hydroxylamine, hypoxanthine, margaric acid, meso-erythritol, monostearin, N-acetylglutamine, pelargonic acid, paraxanthine, phenylpyruvic acid, putrescine, pyridoxamine, pyruvic oxime, ribulose, sarcosine, sorbitol, sorbose, stearic acid, sucrose, trehalose, tyramine, and uric acid in the serum sample is conducted in the detection, and
quality of the serum sample based on a number of times by which the serum sample is subjected to freezing and thawing is evaluated based on intensity of the molecule acquired by the detection.

18. The method for evaluating a sample according to claim 13, wherein
detection of at least one molecule selected from the group consisting of 2-aminobutyric acid, 4-hydroxyproline, S-adenosylhomocysteine, asparagine, aspartic acid, acetylcarnitine, adenine, adenosine, adenosine monophosphate, adenosine 3′,5′-cyclic monophosphate, alanine, allantoin, argininosuccinic acid, arginine, isoleucine, inosine, uridine, ornithine, carnitine, carnosine, xanthine, kynurenine, guanosine, guanosine 3′,5′-cyclic monophosphate, glycine, glutamic acid, creatinine, creatine, cholic acid, succinic acid, choline, cystine, cysteine, cytidine, citrulline, dimethylglycine, serine, serotonin, tyrosine, dopa, dopamine, tryptophan, threonine, nicotinamide, valine, pantothenic acid, histidine, asymmetric dimethylarginine, hypoxanthine, phenylalanine, proline, homocysteine, methionine, methionine sulfoxide, malic acid, leucine, symmetric dimethylarginine, and uric acid in the serum sample by liquid chromatography/mass spectrometry is conducted.

19. The method for evaluating a sample according to claim 18, wherein
detection of at least one molecule selected from the group consisting of adenosine, adenosine 3′,5′-cyclic monophosphate, allantoin, aspartic acid, carnosine, choline, cytidine, dopa, glutamic acid, guanosine, guanosine 3′,5′-cyclic monophosphate, hypoxanthine, inosine, malic acid, nicotinamide, ornithine, S-adenosylhomocysteine, uridine, xanthine, arginine, argininosuccinic acid, cysteine, methionine sulfoxide, serine, succinic acid, asparagine, proline, histidine, pantothenic acid, isoleucine, leucine, dopamine, and glycine in the serum sample is conducted in the detection, and
quality of the serum sample based on time from when the blood is collected until the blood is subjected to centrifugation is evaluated based on intensity of the molecule acquired by the detection.

20. The method for evaluating a sample according to claim 18, wherein
detection of at least one molecule selected from the group consisting of adenine, adenosine, adenosine monophosphate, argininosuccinic acid, carnosine, cystine, cytidine, glutamic acid, guanosine, guanosine 3′,5′-cyclic monophosphate, inosine, malic acid, S-adenosylhomocysteine, serotonin, adenosine 3′,5′-cyclic monophosphate, allantoin, aspartic acid, cysteine, hypoxanthine, methionine sulfoxide, proline, and xanthine in the serum sample is conducted in the detection, and
quality of the serum sample based on time from when centrifugation of the blood is conducted until serum obtained by the centrifugation is isolated is evaluated based on intensity of the molecule acquired by the detection.

21. The method for evaluating a sample according to claim 18, wherein
detection of at least one molecule selected from the group consisting of adenine, adenosine, adenosine 3′,5′-cyclic monophosphate, adenosine monophosphate, allantoin, carnosine, creatine, cysteine, cystine, cytidine, guanosine 3′,5′-cyclic monophosphate, hypoxanthine, inosine, kynurenine, methionine sulfoxide, succinic acid, uridine, xanthine, 2-aminobutyric acid, 4-hydroxyproline, alanine, arginine, argininosuccinic acid, asparagine, asymmetric dimethylarginine, carnitine, cholic acid, choline, citrulline, creatinine, dimethylglycine, dopa, glycine, guanosine, histidine, homocysteine, isoleucine, leucine, methionine, nicotinamide, S-adenosylhomocysteine, serine, symmetric dimethylarginine, threonine, tryptophan, tyrosine, uric acid, acetylcarnitine, aspartic acid, glutamic acid, malic acid, ornithine, pantothenic acid, phenylalanine, proline, serotonin, and valine in the serum sample is conducted in the detection, and
quality of the serum sample based on a number of times by which the serum sample is subjected to freezing and thawing is evaluated based on intensity of the molecule acquired by the detection.

22. An analysis method, which comprises:
conducting evaluation of a serum sample by the method for evaluating a sample according to claim 13; and
conducting analysis of a serum sample based on the evaluation.

23. A method for detecting a degraded sample, which comprises:
obtaining a serum sample prepared from human blood; and
conducting detection of at least one molecule selected from the group consisting of 1,6-anhydroglucose, 1-hexadecanol, 2-aminooctanoic acid, 2-aminobutyric acid, 2-ketoisovaleric acid, 2-hydroxyglutaric acid, 2-hydroxypyridine, 3-aminoisobutyric acid, 3-aminopropionic acid, β-alanine, 3-indolepropionic acid, 3-sulfinoalanine, 3-hydroxyanthranyl acid, 3-hydroxyisovaleric acid, 3-hydroxypyruvic acid, 3-hydroxypropionic acid, 3-phenyllactic acid, 4-hydroxyphenyllactic acid, 4-hydroxyproline, 5-hydroxymethyl-2-furancarboxylic acid, N6-acetyllysine, N-acetylglutamine, N-acetylserine, S-adenosylhomocysteine, aconitic acid, adipic acid, ascorbic acid, asparagine, aspartic acid, acetylcarnitine, acetylglycine, acetoacetic acid, azelaic acid, adenine, adenosine, adenosine monophosphate, adenosine 3′,5′-cyclic monophosphate, arachidonic acid, alanine, allantoin, argininosuccinic acid, arginine, allose, benzoic acid, isoleucine, inositol, inosine, uracil, uridine, eicosapentaenoic acid, erythrulose, octadecanol, ornithine, oleamide, cadaverine, cabroic acid, galacturonic acid, carnitine, carnosine, xanthine, xylitol, xylulose, xylose, kynurenine, guanosine, guanosine 3′,5′-cyclic monophosphate, glyoxylic acid, glycolic acid, glycine, glycerol-3-phosphate, glucosamine, gluconic acid, glutamic acid, glutaric acid, creatinine, creatine, cholic acid, succinic acid, choline, sarcosine, cystine, cysteine, cytidine, citramalic acid, citrulline, dihydrouracil, dihydroxyacetone phosphate, dimethylglycine, oxalic acid, scyllo-inositol, sucrose, stearic acid, serine, serotonin, sorbitol, sorbose, tyramine, tyrosine, decanoic acid, dopa, dopamine, docosahexaenoic acid, tryptophan, threonine, threonic acid, trehalose, nicotinamide, paraxanthine, valine, pantothenic acid, histidine, asymmetric dimethylarginine, hydroxylamine, hypoxanthine, hypotaurine, pyridoxamine, pyruvic oxime, pyruvic acid, phenylalanine, phenylpyruvic acid, phenylbutyric acid, psicose, putrescine, proline, pelargonic acid, boric acid, homocysteine, margaric acid, maleic acid, myo-inositol, myristic acid, meso-erythritol, methionine, methionine sulfoxide, monostearin, lactitol, lactose, ribitol, ribulose, ribose, ribonic acid, ribonic acid lactone, malic acid, leucine, benzoic acid, symmetric dimethylarginine, and uric acid in the serum sample.

24. A marker for detecting a degraded serum sample, which comprises at least one molecule selected from the group consisting of 1,6-anhydroglucose, 1-hexadecanol, 2-aminooctanoic acid, 2-aminobutyric acid, 2-ketoisovaleric acid, 2-hydroxyglutaric acid, 2-hydroxypyridine, 3-aminoisobutyric acid, 3-aminopropionic acid, β-alanine, 3-indolepropionic acid, 3-sulfinoalanine, 3-hydroxyanthranyl acid, 3-hydroxyisovaleric acid, 3-hydroxypyruvic acid, 3-hydroxypropionic acid, 3-phenyllactic acid, 4-hydroxyphenyllactic acid, 4-hydroxyproline, 5-hydroxymethyl-2-furancarboxylic acid, N6-acetyllysine, N-acetylglutamine, N-acetylserine, S-adenosylhomocysteine, aconitic acid, adipic acid, ascorbic acid, asparagine, aspartic acid, acetylcarnitine, acetylglycine, acetoacetic acid, azelaic acid, adenine, adenosine, adenosine monophosphate, adenosine 3′,5′-cyclic monophosphate, arachidonic acid, alanine, allantoin, argininosuccinic acid, arginine, allose, benzoic acid, isoleucine, inositol, inosine, uracil, uridine, eicosapentaenoic acid, erythrulose, octadecanol, ornithine, oleamide, cadaverine, cabroic acid, galacturonic acid, carnitine, carnosine, xanthine, xylitol, xylulose, xylose, kynurenine, guanosine, guanosine 3′,5′-cyclic monophosphate, glyoxylic acid, glycolic acid, glycine, glycerol-3-phosphate, glucosamine, gluconic acid, glutamic acid, glutaric acid, creatinine, creatine, cholic acid, succinic acid, choline, sarcosine, cystine, cysteine, cytidine, citramalic acid, citrulline, dihydrouracil, dihydroxyacetone phosphate, dimethylglycine, oxalic acid, scyllo-inositol, sucrose, stearic acid, serine, serotonin, sorbitol, sorbose, tyramine, tyrosine, decanoic acid, dopa, dopamine, docosahexaenoic acid, tryptophan, threonine, threonic acid, trehalose, nicotinamide, paraxanthine, valine, pantothenic acid, histidine, asymmetric dimethylarginine, hydroxylamine, hypoxanthine, hypotaurine, pyridoxamine, pyruvic oxime, pyruvic acid, phenylalanine, phenylpyruvic acid, phenylbutyric acid, psicose, putrescine, proline, pelargonic acid, boric acid, homocysteine, margaric acid, maleic acid, myo-inositol, myristic acid, meso-erythritol, methionine, methionine sulfoxide, monostearin, lactitol, lactose, ribitol, ribulose, ribose, ribonic acid, ribonic acid lactone, malic acid, leucine, benzoic acid, symmetric dimethylarginine, and uric acid.
